(19) United States
(12) Patent Application Publication
(10) Pub. No.: US 2008/0194485 A1
(43) Pub. Date: Aug. 14, 2008

(54) Detection and Use of Prolylcarboxypeptidase

(75) Inventors: Tamas Horvath, New Haven, CT (US); Sabrina Diano, New Haven, CT (US); Qian Gao, New Haven, CT (US); Craig H. Warden, Davis, CA (US)

Correspondence Address: MORGAN LEWIS & BOCKIUS LLP, 1111 PENNSYLVANIA AVENUE NW, WASHINGTON, DC, 20004, US

(73) Assignees: YALE UNIVERSITY, New Haven, CT (US); THE REGENTS OF THE UNIVERSITY OF CALIFORNIA, Oakland, CA (US)

(21) Appl. No.: 11/597,676

(22) PCT Filed: May 23, 2005
(86) PCT No.: PCT/US05/18191

§ 371 Date: Mar. 31, 2008

Related U.S. Application Data
(60) Provisional application No. 60/573,146, filed on May 21, 2004.

(51) Int. Cl. A61K 38/10; A61P 7/12; A61P 3/04

(52) U.S. Cl. 514/14

(57) ABSTRACT

The present invention relates to weight control, control of body fat and food intake, and provides useful methods for treating, inter alia, obesity, diabetes, and conditions, diseases, and disorders relating thereto.

### CROSS REFERENCE TO RELATED APPLICATIONS

This application is entitled to priority, pursuant to 35 U.S.C. §119(e), to U.S. provisional patent application No. 60/573,146, which was filed on May 21, 2004, which is incorporated herein in its entirety.

### STATEMENT REGARDING FEDERALLY SUPPORTED RESEARCH OR DEVELOPMENT

This research was supported in part by U.S. Government funds (DK52581, DK61619, HL52779, HL57346, and HL65194) and the U.S. Government may therefore have certain rights in the invention.

### BACKGROUND OF THE INVENTION

Obesity is arguably the greatest public health threat in modern Western society, and it is an increasing threat throughout the world. A recent Surgeon General's report underscores the impact of obesity on human health. According to the report, approximately 61% of adults in the United States are overweight or obese, and the prevalence of overweight children and adolescents has doubled in the past two decades. The estimated economic burden of obesity to the United States alone is about $117 billion annually, and obesity is associated with an estimated 300,000 deaths per year, resulting from such disorders as hypertension, dyslipidemia, diabetes, stroke, gallbladder disease, cardiovascular disease, osteoarthritis, hypercholesterolemia, sleep apnea, respiratory problems, cancer, stroke and many other disorders.

In light of the health dangers attributed to obesity, many treatments, both pharmacological and non-pharmacological, have been developed to combat this enormous problem. Pharmacological methods to control weight have targeted a spectrum of physiological processes. Central nervous system (CNS) appetite suppressants interact with catecholaminergic receptors in the brain stem or regulate available serotonin levels. Drawbacks to these agents include possible addiction and numerous side effects including nervousness, insomnia, drowsiness, depression, nausea and lassitude.

Another class of pharmacologic agents for weight control promotes malabsorption of fats and carbohydrates through inhibition of digestive enzymes. Amylase, glycosidase and lipase inhibitors have been isolated from bacterial or fungal sources, and have been used to prevent the absorption of fats and carbohydrates in the digestive tract. A major problem with these agents is that it is virtually impossible to maintain physiological levels of these inhibitors that can effectively inhibit gastrointestinal enzymes, and therefore absorption. Additionally, the use of these inhibitors often leads to compensatory cravings for other foods. As an example, subjects taking a lipase inhibitor will often consume more carbohydrates to compensate for the loss of fat absorption in the diet, thereby negating any weight control benefits.

Another type of weight control agents is non-caloric, non-nutritive dietary substitutes, including saccharine, aspartame, and sucrose polyester (a fat substitute). The sucrose substitutes, saccharine and aspartame, have been linked to hyperphagia in order to compensate for the loss of calories from naturally occurring sucrose, and therefore may not help control weight. Furthermore, these are only sugar substitutes, and do not impact the role of fat consumed in the diet. Sucrose polyester is a sucrose bound to varying numbers and lengths of fatty acid chains. The size and complexity of the fatty acid chains prevent it from being absorbed, but also binds many fat-soluble vitamins, leading to vitamin deficiencies. Further, sucrose polyester has been associated with severe and unpredictable gastrointestinal instability and fecal incontinence.

In addition to the methods directed towards controlling obesity using pharmacological methods, a great deal of research has been conducted to elucidate the underlying genetic and biochemical mechanisms of obesity. Many human genes have been linked directly to obesity (Zhang et al., 1994, Nature 372:425-432; Deng et al., 2002, Am. J. Hum. Genet. 70:1138-1151), or to obesity susceptibility (Frougel and Boutin, 2001, Exp. Biol. Med. 226:991-996). Additionally, many of the genes involved in obesity have also been implicated in diabetes mellitus (Coleman et al., 1978, Diabetologia 14:141-148)

Monogenic human obesity syndromes have validated obesity genes expressed in the hypothalamus that involve melanocortin signaling. Melanocortins (MCs) are peptides cleaved from a common precursor, pro-opiomelanocortin (POMC). The central melanocortin system plays a critical role in the homeostatic regulation of body weight (Cone, 1999; Fan et al., 1997; Huszar et al., 1997; Mizuno and Mobbs, 1999; Butler et al., 2000). Reduced expression of hypothalamic POMC is associated with obesity syndromes caused by mutations in the leptin receptor (Mizuno et al., 1998; Kim et al., 2000), or other genes (tubby, Nhlh2, etc.) (Guan et al., 1998; Good et al., 1997); by hypothalamic damage (Bergen et al., 1998); and perhaps most common, by aging (Mobbs et al., 2001). That reduced hypothalamic POMC mRNA could contribute to the obese phenotypes in these models is suggested by the observation that mutations in the POMC gene cause obesity in mice (Yaswen et al., 1999) and humans (Krude et al., 1998). However, it is still unclear whether normalization of central POMC tone can reverse obese phenotypes.

The prohormone POMC is processed by proteases to produce several peptide hormones including α-melanocyte stimulating hormone (α-MSH). α-MSH is a major regulator of feeding and body weight homeostasis. α-MSH inhibits food intake by binding to the melanocortin receptors 3 and 4 (MC3R and MC4R) (Vaisse et al., 2000). Mutations in POMC that prevent production of α-MSH cause monogenic human obesity (Krude et al., 1998). Mutations in carboxypeptidase E prevent processing of many prohormones, including POMC, and cause obesity (Naggert et al., 1995; Duhl et al., 1994; Jackson et al., 1997; Huszar et al., 1997). Mutations in MC4R may be found in 1 to 5% of all morbidly obese individuals. Mutations in all these genes also cause obesity syndromes in mice.

Despite the recent advances that have been made in understanding the causes for obesity, it remains a largely intractable disorder. It would be useful, therefore, to provide efficacious methods and compositions for the prevention, treatment and amelioration of obesity and obesity-related disorders.

### BRIEF SUMMARY OF THE INVENTION

Prolylcarboxypeptidase (PRCP) is an evolutionarily conserved protease that specifically cleaves peptides with a penultimate proline residue (---px), including α-melanocyte stimulating hormone (α-MSH), a critical regulator of energy homeostasis.

In humans, PRCP nucleotide polymorphism was linked to a metabolic phenotype. In mice, a chromosome 7 congenic mouse model containing only 0.5 centiMorgans of BALB/c donor DNA was significantly leaner than the background C57BL/6Byr strain. PRCP resides in the BALB/c region. To identify the prolylcarboxypeptidase gene (Prcp), positional genetics was used in a congenic mouse line containing BALC/c genome DNA on a C57BL/6J background. Microarray analysis of all 97 genes in the maximal donor region with RNA from 4 tissues revealed no significant genomewide genotype differences, but real time PCR revealed a significant genotype effect on PRCP mRNA in brain, even though PRCP is in a haplotype shared between BALB/c and C57BL/6J. There was a 1.7-fold greater expression in the whole brains of congenic mice than in the background C57BL/6J mice (Table 5). This table shows differential expression measured with 4 separate assays for Prcp and normalized for expression of 4 separate control genes.

In situ hybridization shows that the PRCP gene is expressed in hypothalamic regions associated with feeding regulation, including arcuate (ARC) nucleus and ventral medial hypothalamus (VMH). This location suggests a close association between brain PRCP and α-MSH-producing melanocortin cells. PRCP immunopositive neurons in the hypothalamus of non-human primate were also detected confirming an evolutionarily conserved system.

The anorectic neuropeptide α-MSH, the effecter of POMC feeding neurons inhibiting food intake through melanocortin receptor 4 (MC4), is a potential substrate of PRCP because α-MSH contains a penultimate proline residue: “SYSMEHFRWGKPV” (SEQ ID NO: 1). It is shown that inhibiting. PRCP should increase anorexigenic tone and suppress food intake and weight gain. In contrast to full length α-MSH, the degradation product of α-MSH catalyzed by PRCP has neither neuromodulatory nor anorexigenic effects.

Conversely, inhibition of PRCP activity by protease inhibitors results in suppressed food intake in wild type as well as in genetically obese (ob/ob) mice. Administration of a selective PRCP inhibitor, t-butyl carbamate (BOC)-prolyl prolinal (B-PP), reduced food intake regardless of central or peripheral administration. Food intake reduction was evident even in genetically obese, ob/ob mice. Administration of another PRCP inhibitor, N-benzyloxycarbonyl-prolyl-prolinal (Z-PP), also reduced food intake in after peripheral administration to ob/ob mice. In any of the cases described, food intake returned to control levels after cessation of drug administration, which was not followed by rebound feeding.

Inactivation of Prcp gene in mice unmasked food intake inhibition by peripheral α-MSH administration which was not seen in wild type littermates. These observations reveal PRCP as a target for further obesity drug development.

### BRIEF DESCRIPTION OF THE DRAWINGS

For the purpose of illustrating the invention, there are depicted in the drawings certain embodiments of the invention. However, the invention is not limited to the precise arrangements and instrumentalities of the embodiments depicted in the drawings.

FIG. 1 is a graph depicting the catalytic activity of PRCP on α-MSH, MSH₁₋₁₂ and angiotensinogin II (AgII).

Panel A: Increasing concentration of α-MSH, MSH1-12, or AgII (0.001-1 mM) were incubated with 8 nM of recombinant PRCP51 at 37° C. in microtiter plate cuvette wells with preabsorbed HK and containing 20 nM PK. The liberation of paranitroanilide (pNA) from the S2302 by the formed plasma kallikrein in the presence of the peptide was measured at 405 nm and the results are expressed as residual formed plasma kallikrein activity. The data represent the mean±SEM of three independent measurements.

Panel B: Real time PCR of DNA from mouse tail or cells for lacZ. The probes that were used for these studies are indicated in Table 4. The curves for lacZ DNA shown are for the wild type (ES) or PRCP gene trapped PRCP cell line (KST302), respectively. The curves for PRCP^{-/-} or PRCP^{+/+} indicates PCR of tail DNA from presumed KO or littermate wild type mouse, respectively. The figure is a representative real time PCR on a single mouse or cell DNA sample.

Panel C: RNA from kidney from PRCP^{-/-} or PRCP^{+/+} mice or ES or KST302 cells were subjected to reverse transcription, real time PCR using probes described in Table 4. Primers for Kidney RNA real time PCR are described in Table 4. Amplification plots show the fluorescence plotted against cycle number for the reverse transcribed RNA around the insertional mutation site using sense primers to exon 5 of PRCP and antisense primers to the TM region of pGT1.8 vector and a probe common to the contiguous sequence of exon 5 with the splicing acceptor (SA) region of pGT1.8TM vector (Table 4). The figure is a representative study of the mouse kidney RNA from a wild type and KO animal shown in Panel B.

Panel D: RNA from kidney from PRCP^{-/-} or PRCP^{+/+} mice were subjected to reverse transcription, real time PCR using probes described in Table 4. Amplification plots show the fluorescence of a probe that spans the 3′ region of exon 5 and splice acceptor region of the vector of the cDNA using various combinations of sense primers specific to exons 1 or 5 of PRCP and antisense primers to the TM region of pGT1.8 vector or exons 8, 9, or 10 of PRCP.

FIG. 2 is an image of In situ hybridization showing that the PRCP gene is expressed in hypothalamic regions.

Panel A: Dark blue beta galactosidase labeling representing LacZ expression in the place of PRCP gene in cells of the hypothalamus. Most labeled cells are in an area in the vicinity of the dorsomedial hypothalamic nucleus (DMH), perifornical region (pf) lateral hypothalamus (LH) and zona incerta (ZI). Few labeled cells are also visible in the arcuate nucleus (ARC) of the mediobasal hypothalamus. Fornix, III: third ventricle.

Panel B: Corresponding to the LacZ expression shown on panel A, in situ hybridization for PRCP in wild type animals resulted in labeled cells (black dots representing digoxigenin-labeling of anti sense mRNA probe) in the DMH, pf, LH and ZI with few labeled cells also present in the ARC.

Panels C-F: Double labeling for beta galactosidase (LacZ) and melanin concentrating hormone (MCH), hypocretin (Hcrt) and pro-opiomelanocortin (POMC) revealed extensive coexpression of PRCP and MCH in the lateral hypothalamus-perifornical region (C) and zona incerta (D), and PRCP and Hcrt in the lateral hypothalamus-perifornical region (E). Few cells expressing POMC were also found to express LacZ (representing PRCP) in the arcuate nucleus. Red arrows indicate double labeled cells, black arrows point to single labeled LacZ expressing cells and black arrowheads indicate single labeled MCH, Hcrt or POMC neurons.

FIG. 3A is a graph showing the effect of intracerebral ventrical (ICV) injection of 2.5 mg of α-MSH and α-MSH₁₋₁₂ on food intake compared to saline control.

FIG. 3B shows the electrophysiologic results of α-MSH and α-MSH₁₋₁₂ on GFP POMC neurons of the arcuate nucleus of the hypothalamus.

FIG. 3C is a graph showing the effect of intracerebral ventrical (ICV) administration of vehicle and 0.9 mg B-PP on food intake of fasted rats.

FIG. 3D is a graph showing the percentage of food intake in ob/ob mice injected intraperitoneally with vehicle (open bars) and 400 μg of B-PP (black bars). *P<0.05

FIG. 3E is a graph showing the percentage of food intake in ob/ob mice injected intraperitoneally with vehicle (open bars) and 400 μg of Z-PP (black bars). *P<0.05

FIG. 4A shows the percentage of food intake after intraperitoneal (IP) injection of 200 nmol of α-MSH in wild type (WT) or PRCP KO mice compared to saline control animals.

FIG. 4B shows food intake in grams after intraperitoneal (IP) injection of 200 nmol of α-MSH in wild type (WT) or PRCP KO mice compared to saline control animals. * P<0.05

### DETAILED DESCRIPTION OF THE INVENTION

The present invention is partly based on the discovery that inhibition of PRCP decreases food intake, body fat, and affects physical activity and metabolic rate. These unexpected results demonstrate that inhibition of PRCP is a treatment for a disease, disorder or condition mediated by food intake, and the like. Such a disease, disorder or condition includes, but is not limited to, obesity, hypertension, dyslipidemia, diabetes, stroke, gallbladder disease, cardiovascular disease, osteoarthritis, rheumatoid arthritis, hypercholesterolemia, stable angina, unstable angina, arthrosclerosis, sleep apnea, respiratory problems, cancer, stroke, and the like.

The present invention also includes methods of identifying additional compounds that inhibit PRCP for affecting a disease, disorder or condition mediated by increased body fat, decreased physical activities, decreased metabolic activity, and the like.

### Definitions

As used herein, each of the following terms has the meaning associated with it in this section.

The articles “a” and “an” are used herein to refer to one or to more than one (i.e. to at least one) of the grammatical object of the article. By way of example, “an element” means one element or more than one element.

“Prolylcarboxypeptidase” and “PRCP” as used herein refer to an evolutionarily conserved protease that specifically cleaves peptides with a penultimate proline residue (---px), including α-melanocyte stimulating hormone (α-MSH), a critical regulator of energy homeostasis. PRCP is also known by various other names by those skilled in the art including, but not limited to prolylcarboxypeptidase Pro-X (PRCP-Pro-X), angiotensinase C, Pro-x carboxypeptidase, Lysosomal Pro-X carboxypeptidase precursor, Proline carboxypeptidase and Lysosomal carboxypeptidase C.

“PRCP activity” is used herein to refer the function of the prolylcarboxypeptidase, including, but not limited to, cleaving the peptides with a penultimate proline residue (---px, wherein “---” designates the preceding amino acids of the protein, “p” designates a proline residue, and “x” designates any amino acid) resulting a change in the peptides function, and the like.

“PRCP inhibiting” is used herein to refer to detectably decreasing the activity, function and/or expression of PRCP.

By the term “applicator,” as the term is used herein, is meant any device including, but not limited to, a hypodermic syringe, a pipette, and the like, for administering the inhibitor of PRCP of the invention to a mammal.

The term “inhibitor” is used herein to refer to a composition of matter which when administered to a mammal such as a human, inhibits a biological activity attributable to the level or presence of an endogenous compound in the mammal.

The term “weight loss” is used herein to refer to a detectable decrease of body mass in an animal compared to the mass of the animal at a previous time.

As used herein, “an effective amount of an PRCP inhibitor” means an amount of a compound which effects a detectable decrease in the expression, activity, or both, of PRCP in a cell, an animal, or both, compared with the level of expression, activity, or both, of PRCP in an otherwise identical cell or animal to which the compound is not administered, or in the same cell or animal prior to administration of the compound.

As used herein, the term “antisense nucleic acid molecule” means a nucleic acid polymer, at least a portion of which is complementary to a nucleic acid, which is present in a normal cell or in an affected cell. The antisense nucleic acid molecules of the invention preferably comprise between about ten and about one hundred nucleotides. Most preferably, the antisense nucleic acid molecules comprise between about fifteen and about fifty nucleotides. The antisense oligonucleotides of the invention include, but are not limited to, phosphorothioate oligonucleotides and other modifications of oligonucleotides. Methods for synthesizing oligonucleotides, phosphorothioate oligonucleotides, and otherwise modified oligonucleotides are well known in the art (U.S. Pat. No. 5,034,506; Nielsen et al., 1991, Science 254: 1497).

As used herein, the term “obesity-related disorder” refers to any condition where the accumulation of excess fat is a risk factor for developing health complications. Over time, weight loss in obese individuals may reduce a number of health risks. Studies looking at the effects of weight-loss medication treatment on obesity-related health risks have found that some agents lower blood pressure, blood cholesterol, and triglycerides (fats) and decrease insulin resistance (the body's inability to use blood sugar) via a reduction in weight.

For purposes of the present invention, obesity-related disorders include, but are not limited to, hypertension, dyslipidemia, diabetes, stroke, gallbladder disease, cardiovascular disease, osteoarthritis, rheumatoid arthritis, hypercholesterolemia, stable angina, unstable angina, sleep apnea, respiratory problems, cancer, stroke hyerinsulinemia, Syndrome X, hypercholesterolemia, hyperlipoproteinemia, hypertriglyceridemia, atherosclerosis, diabetic renal disease and many other disorders. Thus, the reduction of excess fat by the methods and compositions of the present invention also helps to prevent or to treat any health complication arising from the condition of having the excess fat.

By the term “specifically binds” as used herein, is meant an antibody which recognizes and binds PRCP, but does not substantially recognize or bind other molecules in a sample.

“Physical activity” is used herein to refer to any detectable movement, action, and/or alertness in an animal.

“Affecting physical activity” in an animal encompasses mediating a detectably higher or lower level of movement, action, and/or alertness in the animal, whereas decreasing physical activity in an animal is achieving a lower level of movement, action, and/or alertness.

“Metabolic rate” is used herein to refer to the use of calories or another energy source in an animal as assessed over a period of time. Hence, an increased metabolic rate is a higher level of the use of calories or another energy source by an animal over a period of time compared with the level of use of calories by an otherwise identical animal over the same period of time under substantially similar or identical conditions.

### Description

I. Methods

A. Method of Treating and/or Preventing a Disease Disorder or Condition

The present invention is based, in part, on the novel discovery that PRCP plays a significant role in weight control in a mammal. As demonstrated by the data disclosed herein, inhibition of PRCP can induce weight loss, decrease body fat, affect appetite, decrease food intake, both treat and prevent obesity. The data disclosed here demonstrate that PRCP is involved in the signal transduction pathway for food intake and weight control. Thereby, the present invention discloses methods to treat various diseases relating to food intake and weight control, including, but not limited to, obesity, hypertension, dyslipidemia, diabetes, stroke, gallbladder disease, cardiovascular disease, osteoarthritis, hypercholesterolemia, sleep apnea, respiratory problems, cancer, stroke and many other disorders.

The present invention includes a method of inducing weight loss in an animal. This is because, as demonstrated by the data disclosed elsewhere herein, inhibition of PRCP, whether its expression, biological activity, or both, is inhibited, mediates a variety of physiological responses, including, but not limited to, decreased body fat, decreased food intake, increased physical activities, and increased metabolic rate. Thus, inhibiting PRCP can be used to treat a wide variety of diseases, disorders or conditions where decreasing body fat and food intake would provide a therapeutic benefit to an animal. Accordingly, one skilled in the art would appreciate, based upon the disclosure provided herein, that inhibiting PRCP provides an important, novel therapeutic for treatment of, inter alia, obesity, hypertension, dyslipidemia, diabetes, stroke, gallbladder disease, cardiovascular disease, osteoarthritis, hypercholesterolemia, sleep apnea, respiratory problems, cancer, stroke and many other disorders, and the like. More particularly, PRCP can be inhibited thereby mediating weight loss in an animal.

The skilled artisan would appreciate, based upon the disclosure provided herein, that an animal encompasses a bird, a fish, and a mammal, where the mammal includes, but is not limited to, a rodent, a cow, a pig, a sheep, a buffalo, a beefalo (a cow/buffalo hybrid animal), a bison, a deer, a goat, and a human. One skilled in the art, armed with the teachings provided herein, would appreciate that the animal comprises PRCP, which can be inhibited.

An inhibitor of PRCP is administered to the animal thereby decreasing PRCP and providing a therapeutic benefit. The skilled artisan would appreciate, based upon the disclosure provided herein, that PRCP can be inhibited using a wide plethora of techniques well-known in the art or to be developed in the future. That is, the invention encompasses inhibiting PRCP expression, e.g., inhibition of transcription and/or translation. This is because, as demonstrated by the data disclosed elsewhere herein, knocking-out the nucleic acid encoding PRCP, such that the nucleic acid was not transcribed or translated, mediated a variety of effects, including, but not limited to, decreased food intake, loss of weight, and a decrease in body fat. Thus, inhibiting PRCP includes, but is not limited to, inhibiting translation and/or transcription of a nucleic acid encoding the protein.

Further, the routineer would understand, based upon the disclosure provided elsewhere herein, that inhibition of PRCP includes, but is not limited to, inhibiting the biological activity of the molecule. This is because, as the data disclosed elsewhere herein demonstrate, inhibition of PRCP activity using a PRCP inhibitor (e.g., B-PP) mediated a decrease in food intake and body fat. These data indicate that inhibition of PRCP activity provides a therapeutic benefit for treatment of a disease, such as, but not limited to, obesity, or diabetes.

The skilled artisan would understand that an inhibitor of PRCP encompasses, but is not limited to, an inhibitor of a carboxypeptidase, including, but not limited to, t-butyl carbamate (BOC)-prolyl prolinal (B-PP), N-benzyloxycarbonyl-prolyl-prolinal (Z-PP), diisopropyl fluorophosphate, PMSF, antipain, leupeptin, corn trypsin, and mercuric chloride (high concentrations).

One skilled in the art, based upon the disclosure provided herein, would appreciate that PRCP inhibition can be mediated by using, inter alia, an antibody, an antisense nucleic acid, a siRNA, an aptamer, a ribozyme, a small molecule, a peptidomimetic, and a chemical compound, either known or to be developed, which inhibits PRCP expression, activity, or both. That is, the invention encompasses using a PRCP inhibiting compound such as, but not limited to, B-PP and Z-PP. This is because, as is demonstrated by the data disclosed elsewhere herein, inhibition of PRCP expression and/or activity leads to reduced food intake, reduced body weight, and the like.

Further, one of skill in the art would, when equipped with this disclosure and the methods described herein, recognize that PRCP inhibitors include such inhibitors as discovered in the future, as could be established by well known criteria in the art of pharmacology, and those identified in light of the physiological results of inhibition of PRCP as described in detail herein. Therefore, the present invention is not limited in any way to the PRCP inhibitors described herein, but includes those PRCP inhibitors, and other carboxypeptidase inhibitors, that inhibit, inter alia, PRCP, as are discovered in the future.

Methods of obtaining and generating carboxypeptidase inhibitors are well known to those of ordinary skill in the art. For instance, a carboxypeptidase inhibitor can be isolated from a naturally occurring source. Likewise, a carboxypeptidase inhibitor can be readily synthesized chemically.

An inhibitor of PRCP may be an antibody that specifically binds to PRCP thereby inhibiting the action of PRCP. Antibodies that specifically bind to PRCP are well known to those of ordinary skill in the art (Shariat-Madar et al., 2002, 2004), may be purchased commercially, or can be produced using standard methods (Harlow et al., 1988, Antibodies: A Laboratory Manual, Cold Spring Harbor, N.Y.).

The term “antibody,” as used herein, refers to an immunoglobulin molecule which is able to specifically bind to a specific epitope on an antigen. Antibodies can be intact immunoglobulins derived from natural sources or from recombinant sources and can be immunoreactive portions of intact immunoglobulins. Antibodies are typically tetramers of immunoglobulin molecules. The antibodies in the present invention may exist in a variety of forms including, for example, polyclonal antibodies, monoclonal antibodies, Fv, Fab and F(ab)₂, as well as single chain antibodies and humanized antibodies (Harlow et al., 1999, Using Antibodies: A Laboratory Manual, Cold Spring Harbor Laboratory Press, NY; Harlow et al., 1989, Antibodies: A Laboratory Manual, Cold Spring Harbor, N.Y.; Houston et al., 1988, Proc. Natl. Acad. Sci. USA 85:5879-5883; Bird et al., 1988, Science 242:423-426).

One of skill in the art will appreciate that an antibody can be administered as a protein, a nucleic acid construct encoding a protein, or both. Numerous vectors and other compositions and methods are well known for administering a protein or a nucleic acid construct encoding a protein to cells or tissues. Therefore, the invention includes a method of administering an antibody or nucleic acid encoding an antibody (synthetic antibody) that is specific for PRCP (Sambrook et al., 1989, Molecular Cloning: A Laboratory Manual, Cold Spring Harbor Laboratory, New York; Ausubel et al., 1997, Current Protocols in Molecular Biology, John Wiley & Sons, New York).

By the term “synthetic antibody” as used herein, is meant an antibody which is generated using recombinant DNA technology, such as, for example, an antibody expressed by a bacteriophage as described herein. The term should also be construed to mean an antibody which has been generated by the synthesis of a DNA molecule encoding the antibody and which DNA molecule expresses an antibody protein, or an amino acid sequence specifying the antibody, wherein the DNA or amino acid sequence has been obtained using synthetic DNA or amino acid sequence technology which is available and well known in the art.

One skilled in the art would understand, based upon the disclosure provided herein, that an antibody can be administered such that it inhibits the function of PRCP present in a membrane. Moreover, the invention encompasses administering an antibody that specifically binds with PRCP, or a nucleic acid encoding the antibody, wherein the molecule further comprises an intracellular retention sequence such that antibody binds with the PRCP and prevents its expression in a membrane. Such antibodies, frequently referred to as “intrabodies”, are well known in the art and are described in, for example, Marasco et al. (U.S. Pat. No. 6,004,490) and Beerli et al. (1996, Breast Cancer Research and Treatment 38:11-17). Thus, the invention encompasses methods comprising inhibiting PRCP where the PRCP is present in a cell membrane, as well as methods of inhibiting PRCP comprising inhibiting the PRCP being present in the cell membrane, and such methods as become known in the future.

As noted previously elsewhere herein, the present encompasses inhibiting PRCP by inhibiting expression of a nucleic acid encoding PRCP. Methods for inhibiting the expression of a gene are well known to those of ordinary skill in the art, and include the use of antisense nucleic acid molecules, ribozymes, interference RNA, or aptamers.

Antisense nucleic acid molecules are DNA or RNA molecules that are complementary to some portion of an mRNA molecule. When present in a cell, antisense nucleic acids hybridize to an existing mRNA molecule and inhibit translation into a gene product. Inhibiting the expression of a gene using an antisense nucleic acid molecule is well known in the art (Marcus-Sekura, 1988, Anal. Biochem. 172:289), as are methods to express an antisense nucleic acid molecule in a cell (Inoue, 1993, U.S. Pat. No. 5,190,931).

Antisense nucleic acid molecules can be synthesized and provided to the cell by way of methods well known to those of ordinary skill in the art. Antisense nucleic acid molecules can be synthesized, for example, to be between about 10 and about 100, more preferably between about 15 and about 50 nucleotides long. The synthesis of nucleic acid molecules is well known in the art. The antisense nucleic acid molecule can also be modified to improve biological activity in comparison to unmodified antisense nucleic acid molecules (Tullis, 1991, U.S. Pat. No. 5,023,243).

Inhibition of expression of PRCP can also be achieved by using a ribozyme. Using ribozymes for inhibiting gene expression is well known to those of ordinary skill in the art (Cech et al., 1992, J. Biol. Chem. 267:17479; Hampel et al., 1989, Biochemistry 28: 4929; Altman et al., 1992, U.S. Pat. No. 5,168,053). Ribozymes are catalytic RNA molecules with the ability to cleave other single-stranded RNA molecules. Ribozymes are known to be sequence specific, and can therefore be modified to recognize a specific nucleotide sequence (Cech, 1988, J. Amer. Med. Assn. 260:3030), allowing the selective cleavage of specific mRNA molecules. Given the nucleotide sequence of PRCP is well known in the art, one of ordinary skill in the art can synthesize an antisense polynucleotide or ribozyme without undue experimentation, provided with the disclosure and references incorporated herein.

Another way of inhibition of expression of PRCP is by using RNA interference (RNAi). RNAi is a phenomenon in which the introduction of double-stranded RNA (dsRNA) into a diverse range of organisms and cell types causes degradation of the complementary mRNA. In the cell, long dsRNAs are cleaved into short 21-25 nucleotide small interfering RNAs, or siRNAs, by a ribonuclease known as Dicer. The siRNAs subsequently assemble with protein components into an RNA-induced silencing complex (RISC), unwinding in the process. Activated RISC then binds to complementary transcript by base pairing interactions between the siRNA antisense strand and the mRNA. The bound mRNA is cleaved and sequence specific degradation of mRNA results in gene silencing. For example, the present invention encompasses methods of inhibiting expression of a Prcp gene in a cell in an animal comprising providing at least one ribonucleic acid (RNA) to the cell in an amount sufficient to inhibit the expression of a Prcp gene, wherein the RNA comprises or forms a double-stranded structure containing a first strand consisting essentially of a ribonucleotide sequence which corresponds to a nucleotide sequence of a Prcp gene and a second strand consisting essentially of a ribonucleotide sequence which is complementary to the nucleotide sequence of the Prcp gene, wherein the first and the second ribonucleotide sequences are complementary sequences that hybridize to each other to form said double-stranded structure, and the RNA comprising the double-stranded structure inhibits expression of a Prcp gene. The RNA can be provided to the cell by contacting the cell with the RNA. Alternatively, the RNA can be provided to the cell by synthesizing the RNA in the cell. For further information see, for example, U.S. Pat. No. 6,506,559; WO 00/01846; WO 00/44914; WO 00/44895; WO 00/63364; WO 01/68836; WO 01/75164; WO 02/44321; WO 03/010180; US Patent Application Publication No. 2003/0148519; WO 03/068797; US Patent Application Publication No. 2003/0203868; WO 99/61631; Fire et al., Nature (1998) 391(19):306-311; Timmons et al., Nature (1998) 395:854; Montgomery et al., TIG (1998) 14(7):255-258; David R. Engelke, Ed., RNA Interference (RNAi) Nuts & Bolts of RNAi Technology, DNA Press (2003); Gregory J. Hannon, Ed., RNAi A Guide to Gene Silencing, Cold Spring Harbor Laboratory Press (2003); and Krishnarao Appasani, Ed., RNA Interference Technology, Cambridge University Press (2005), each one of which is specifically and completely incorporated by reference herein. Therefore, the present invention also includes methods of silencing the gene encoding PRCP by using RNAi technology.

Inhibition of PRCP can also be achieved by using an aptamer. Aptamers are nucleic acid molecules having specific binding affinity to molecules through interactions other than classic Watson-Crick base pairing. Aptamers, like peptides generated by phage display or monoclonal antibodies (MAbs), are capable of specifically binding to selected targets and, through binding, block their targets' ability to function. Created by an in vitro selection process from pools of random sequence oligonucleotides, aptamers have been generated for over 100 proteins including growth factors, transcription factors, enzymes, immunoglobulins, and receptors. A typical aptamer is 10-15 kDa in size (30-45 nucleotides), binds its target with sub-nanomolar affinity, and discriminates against closely related targets (e.g., will typically not bind other proteins from the same gene family). A series of structural studies have shown that aptamers are capable of using the same types of binding interactions (e.g., hydrogen bonding, electrostatic complementarity, hydrophobic contacts, and steric exclusion) that drive affinity and specificity in antibody-antigen complexes. Aptamers have a number of desirable characteristics for use as therapeutics (and diagnostics) including high specificity and affinity, biological efficacy, and excellent pharmacokinetic properties. In addition, they offer specific competitive advantages over antibodies and other protein biologics, for example: Speed and control. Aptamers are produced by an entirely in vitro process, allowing for the rapid generation of initial therapeutic leads. In vitro selection allows the specificity and affinity of the aptamer to be tightly controlled and allows the generation of leads against both toxic and non-immunogenic targets. Given the nucleotide sequence of PRCP is well known in the art, one of ordinary skill in the art can synthesize a PRCP-specific aptamer that targets and blocks the function of PRCP.

The inhibitors of PRCP or of PRCP gene expression may be administered singly or in any combination thereof. Further, inhibitors of PRCP may be administered singly or in any combination thereof in a temporal sense, in that they may be administered simultaneously, before, and/or after each other. One of ordinary skill in the art will appreciate the use of PRCP inhibitors or inhibitors of PRCP gene expression to affect weight loss, a treatment of obesity, and will use the inhibitors detailed herein alone or in any combination to effect such results.

Thus, the invention encompasses a method for inducing weight loss where an inhibitor of PRCP expression, activity, or both, is administered to an animal, thereby effecting a decreased food intake and thereby mediating weight in loss by the animal.

Similarly, the present invention encompasses a method for decreasing body fat in an animal. The method comprises administering a PRCP inhibiting amount of a PRCP inhibitor to the animal. This is because, as more fully set forth previously elsewhere herein, the data disclosed herein demonstrate that inhibition of PRCP (expression, activity, or both) mediates a decrease in body fat, thereby decreasing body fat in the animal.

One skilled in the art would appreciate that having a leaner animal, i.e., an animal with decreased body fat, would provide a benefit in that such an animal would be useful in providing a leaner source of meat having a decreased amount of fat. Such animal includes, but is not limited to, a bird, a fish, a rodent, a cow, a pig, a sheep, a buffalo, a beefalo, a bison, a deer, and a goat. Basically, the invention includes producing an animal comprising a decreased body fat content where the animal is used for human consumption.

The invention includes a method for decreasing food intake in a mammal. Basically, the method comprises administering a PRCP inhibiting amount of a PRCP inhibitor to a mammal. This, in turn, decreases food intake in the mammal. This is because, as disclosed elsewhere herein, the data demonstrate that inhibition of PRCP mediates a decrease in the food intake by a mammal compared to an otherwise identical animal to which the inhibitor is not administered or compared with the food intake by the same animal before administration of the PRCP inhibitor. Thus, the invention encompasses a method of decreasing food intake by inhibiting PRCP.

The amount, dosing regimen, and route of administration for inhibiting PRCP in an animal can be readily determined by one skilled in the art and would depend on well-known factors, including, but not limited to, the age and condition of the animal, the weight and body fat content of the animal, and the desired weight and/or body fat content of the animal. The dosage and route of administration can be easily determined as exemplified elsewhere herein using art-recognized models of obesity and diabetes, and therefore, the skilled artisan would understand, based upon the disclosure provided herein, precisely how to inhibit PRCP to practice the methods of the invention.

Similarly, the invention includes a method for affecting appetite in a mammal. Once again, the method comprises administering a PRCP inhibiting amount of a PRCP inhibitor to a mammal, thereby affecting appetite in the mammal. This is because, as discussed previously elsewhere herein, the data disclosed demonstrate that inhibition of PRCP decreases appetite, as indicated by decreased food intake, in a mammal. This method is useful for providing a therapeutic benefit where the mammal is in need of reducing their appetite, such as when, for instance, the mammal is obese and/or suffers from diabetes or any other disease mediated by, or associated with, increased weight.

The invention encompasses a method for treating obesity in a mammal. The method comprises administering a PRCP inhibiting amount of a PRCP inhibitor to a mammal. As demonstrated by the data disclosed herein, including, but not limited to, data demonstrating weight loss by obese mice using an art-recognized model of obesity, by inhibiting PRCP in the mice, inhibiting PRCP inhibits food intake and decreases weight, thereby treating obesity in the mammal.

The present invention includes a method for preventing obesity in a mammal. The method comprises administering a PRCP inhibiting amount of a PRCP inhibitor to a mammal. This is because the data disclosed elsewhere herein demonstrate that inhibiting PRCP not only treats obesity, but also actually prevents obesity in an art-recognized model of obesity. Thus, the skilled artisan would appreciate, based on the teachings provided herein, that the invention includes, but is not limited to, a method for preventing obesity in a mammal.

The present invention further includes a method for affecting physical activity in an animal. The method comprises administering a PRCP inhibiting amount of a PRCP inhibitor to an animal thereby affecting physical activity. This is because, as demonstrated by the data disclosed elsewhere herein, of PRCP affects a number of physiological responses, including, but not limited to increased physical activities. Thus, inhibiting PRCP can be used to treat a wide variety of disorders, diseases, and/or conditions where decreasing body fat, affecting activity, decreasing food intake, would provide a therapeutic benefit to mammal. Therefore, the skilled artisan will appreciate, based on the disclosure provided herein, that surprisingly, inhibition of PRCP provides a novel method for affecting the physical activity of an animal.

One skilled in the art would understand, based upon the disclosure provided herein, that affecting PRCP encompasses both inhibiting and increasing PRCP expression, activity, or both. This is because the skilled artisan would appreciate, once armed with the teachings provided herein, that inhibiting PRCP increases physical activity such that increasing PRCP activity and/or expression can decrease physical activity in an animal. Thus, the present invention includes increasing and decreasing PRCP activity and/or expression thereby either increasing or decreasing physical activity in an animal. Preferably, PRCP activity and/or expression is inhibited thereby increasing physical activity in an animal.

The skilled artisan would further appreciate, once armed with the teachings provided herein, that a method of increasing physical activity can be used to treat a disease or condition mediated by, or associated with, decreased physical activity. Such diseases or conditions include, but are not limited to, depression, narcolepsy, fatigue, and the like.

The present invention includes a method of affecting metabolic rate in an animal. This is because, as demonstrated by the data disclosed elsewhere herein, inhibition of PRCP affects a variety of physiological processes, including, but not limited to, decreased food intake, increased physical activities, and increased metabolic rate. Thus, the data disclosed herein demonstrate that inhibiting PRCP can affect metabolic rate in an animal. Accordingly, one skilled in the art would appreciate, based upon the disclosure provided herein, that inhibiting PRCP provides an important, novel therapeutic for treatment of, inter alia, obesity and diabetes, atherosclerosis, diabetic renal disease, and the like, where affecting the metabolic rate mediates a therapeutic benefit. More particularly, PRCP can be inhibited thereby affecting metabolic rate in an animal; therefore, inhibition of PRCP can be used to treat, inter alia, obesity and other diseases, disorders and conditions related to decreased metabolic rate.

However, the present invention is not limited to increasing the metabolic rate. Rather, the invention encompasses methods for decreasing the metabolic rate where such decrease would provide a benefit. This is because the data disclosed herein demonstrate that inhibiting PRCP mediates an increase in metabolic rate such that increasing PRCP can decrease metabolic rate. Decreasing the metabolic rate in an animal can be used, for instance, where weight gain and/or increased body fat content in the animal is desired or would provide a therapeutic benefit. Conditions and diseases where decreased metabolic rate, increased body fat, and the like, is desirable would be readily apparent to one skilled in the art based upon the disclosure provided herein.

The invention includes a method for increasing physical activity and/or metabolic rate in a mammal. The skilled artisan would appreciate, based upon the teachings provided herein, that PRCP inhibitors can treat physical activity-related diseases or disorders such as narcolepsy, depression, and the like, and that the PRCP inhibitors can be used to treat metabolism-related disease such as obesity.

The invention also encompasses the use of a pharmaceutical composition comprising an appropriate PRCP inhibitor to practice the methods of the invention, where the composition comprises an appropriate PRCP inhibitor, which can be used in an amount sufficient to inhibit PRCP thereby producing a therapeutic effect, and a pharmaceutically acceptable carrier.

The pharmaceutical compositions useful for practicing the invention may be administered to deliver a dose of between 1 ng/kg/day and 100 mg/kg/day.

Pharmaceutical compositions that are useful in the methods of the invention may be administered systemically in oral solid formulations, ophthalmic, suppository, aerosol, and topical or other similar formulations. In addition to the appropriate PRCP inhibitor, such pharmaceutical compositions may contain pharmaceutically acceptable carriers and other ingredients known to enhance and facilitate drug administration. Other possible formulations, such as nanoparticles, liposomes, resealed erythrocytes, and immunologically based systems may also be used to administer an appropriate PRCP inhibitor according to the methods of the invention.

Compounds, which are identified using any of the methods described herein, may be formulated and administered to a mammal for treatment of the diseases disclosed herein are now described.

The invention encompasses the preparation and use of pharmaceutical compositions comprising a compound useful for treatment of the diseases disclosed herein as an active ingredient. Such a pharmaceutical composition may consist of the active ingredient alone, in a form suitable for administration to a subject, or the pharmaceutical composition may comprise the active ingredient and one or more pharmaceutically acceptable carriers, one or more additional ingredients, or some combination of these. The active ingredient may be present in the pharmaceutical composition in the form of a physiologically acceptable ester or salt, such as in combination with a physiologically acceptable cation or anion, as is well known in the art.

As used herein, the term “pharmaceutically acceptable carrier” means a chemical composition with which the active ingredient may be combined and which, following the combination, can be used to administer the active ingredient to a subject.

As used herein, the term “physiologically acceptable” ester or salt means an ester or salt form of the active ingredient which is compatible with any other ingredients of the pharmaceutical composition, which is not deleterious to the subject to which the composition is to be administered.

The formulations of the pharmaceutical compositions described herein may be prepared by any method known or hereafter developed in the art of pharmacology. In general, such preparatory methods include the step of bringing the active ingredient into association with a carrier or one or more other accessory ingredients, and then, if necessary or desirable, shaping or packaging the product into a desired single- or multi-dose unit.

Although the descriptions of pharmaceutical compositions provided herein are principally directed to pharmaceutical compositions, are suitable for ethical administration to humans, it will be understood by the skilled artisan that such compositions are generally suitable for administration to animals of all sorts. Modification of pharmaceutical compositions suitable for administration to humans in order to render the compositions suitable for administration to various animals is well understood, and the ordinarily skilled veterinary pharmacologist can design and perform such modification with merely ordinary, if any, experimentation. Subjects to which administration of the pharmaceutical compositions of the invention is contemplated include, but are not limited to, humans and other primates, mammals including commercially relevant mammals such as cattle, pigs, horses, sheep, cats and dogs, and birds including commercially relevant birds such as chickens, ducks, geese, and turkeys.

Pharmaceutical compositions that are useful in the methods of the invention may be prepared, packaged, or sold in formulations suitable for oral, rectal, vaginal, parenteral, topical, pulmonary, intranasal, buccal, ophthalmic, intrathecal or another route of administration. Other contemplated formulations include projected nanoparticles, liposomal preparations, resealed erythrocytes containing the active ingredient, and immunologically based formulations.

A pharmaceutical composition of the invention may be prepared, packaged, or sold in bulk, as a single unit dose, or as a plurality of single unit doses. As used herein, a “unit dose” is discrete amount of the pharmaceutical composition comprising a predetermined amount of the active ingredient. The amount of the active ingredient is generally equal to the dosage of the active ingredient which would be administered to a subject or a convenient fraction of such a dosage such as, for example, one-half or one-third of such a dosage.

The relative amounts of the active ingredient, the pharmaceutically acceptable carrier, and any additional ingredients in a pharmaceutical composition of the invention will vary, depending upon the identity, size, and condition of the subject treated and further depending upon the route by which the composition is to be administered. By way of example, the composition may comprise between 0.1% and 100% (w/w) active ingredient.

In addition to the active ingredient, a pharmaceutical composition of the invention may further comprise one or more additional pharmaceutically active agents. Particularly contemplated additional agents include anti-emetics and scavengers such as cyanide and cyanate scavengers.

Controlled- or sustained-release formulations of a pharmaceutical composition of the invention may be made using conventional technology.

A formulation of a pharmaceutical composition of the invention suitable for oral administration may be prepared, packaged, or sold in the form of a discrete solid dose unit including, but not limited to, a tablet, a hard or soft capsule, a cachet, a troche, or a lozenge, each containing a predetermined amount of the active ingredient. Other formulations suitable for oral administration include, but are not limited to, a powdered or granular formulation, an aqueous or oily suspension, an aqueous or oily solution, or an emulsion.

As used herein, an “oily” liquid is one which comprises a carbon-containing liquid molecule and which exhibits a less polar character than water.

A tablet comprising the active ingredient may, for example, be made by compressing or molding the active ingredient, optionally with one or more additional ingredients. Compressed tablets may be prepared by compressing, in a suitable device, the active ingredient in a free-flowing form such as a powder or granular preparation, optionally mixed with one or more of a binder, a lubricant, an excipient, a surface active agent, and a dispersing agent. Molded tablets may be made by molding, in a suitable device, a mixture of the active ingredient, a pharmaceutically acceptable carrier, and at least sufficient liquid to moisten the mixture. Pharmaceutically acceptable excipients used in the manufacture of tablets include, but are not limited to, inert diluents, granulating and disintegrating agents, binding agents, and lubricating agents. Known dispersing agents include, but are not limited to, potato starch and sodium starch glycollate. Known surface-active agents include, but are not limited to, sodium lauryl sulphate. Known diluents include, but are not limited to, calcium carbonate, sodium carbonate, lactose, microcrystalline cellulose, calcium phosphate, calcium hydrogen phosphate, and sodium phosphate. Known granulating and disintegrating agents include, but are not limited to, corn starch and alginic acid. Known binding agents include, but are not limited to, gelatin, acacia, pre-gelatinized maize starch, polyvinylpyrrolidone, and hydroxypropyl methylcellulose. Known lubricating agents include, but are not limited to, magnesium stearate, stearic acid, silica, and talc.

Tablets may be non-coated or they may be coated using known methods to achieve delayed disintegration in the gastrointestinal tract of a subject, thereby providing sustained release and absorption of the active ingredient. By way of example, a material such as glyceryl monostearate or glyceryl distearate may be used to coat tablets. Further by way of example, tablets may be coated using methods described in U.S. Pat. Nos. 4,256,108; 4,160,452; and 4,265,874 to form osmotically controlled release tablets. Tablets may further comprise a sweetening agent, a flavoring agent, a coloring agent, a preservative, or some combination of these in order to provide pharmaceutically elegant and palatable preparation.

Hard capsules comprising the active ingredient may be made using a physiologically degradable composition, such as gelatin. Such hard capsules comprise the active ingredient, and may further comprise additional ingredients including, for example, an inert solid diluent such as calcium carbonate, calcium phosphate, or kaolin.

Soft gelatin capsules comprising the active ingredient may be made using a physiologically degradable composition, such as gelatin. Such soft capsules comprise the active ingredient, which may be mixed with water or an oil medium such as peanut oil, liquid paraffin, or olive oil.

Liquid formulations of a pharmaceutical composition of the invention which are suitable for oral administration may be prepared, packaged, and sold either in liquid form or in the form of a dry product intended for reconstitution with water or another suitable vehicle prior to use.

Liquid suspensions may be prepared using conventional methods to achieve suspension of the active ingredient in an aqueous or oily vehicle. Aqueous vehicles include, for example, water and isotonic saline. Oily vehicles include, for example, almond oil, oily esters, ethyl alcohol, vegetable oils such as arachis, olive, sesame, or coconut oil, fractionated vegetable oils, and mineral oils such as liquid paraffin. Liquid suspensions may further comprise one or more additional ingredients including, but not limited to, suspending agents, dispersing or wetting agents, emulsifying agents, demulcents, preservatives, buffers, salts, flavorings, coloring agents, and sweetening agents. Oily suspensions may further comprise a thickening agent. Known suspending agents include, but are not limited to, sorbitol syrup, hydrogenated edible fats, sodium alginate, polyvinylpyrrolidone, gum tragacanth, gum acacia, and cellulose derivatives such as sodium carboxymethylcellulose, methylcellulose, hydroxypropylmethylcellulose. Known dispersing or wetting agents include, but are not limited to, naturally-occurring phosphatides such as lecithin, condensation products of an alkylene oxide with a fatty acid, with a long chain aliphatic alcohol, with a partial ester derived from a fatty acid and a hexitol, or with a partial ester derived from a fatty acid and a hexitol anhydride (e.g. polyoxyethylene stearate, heptadecaethyleneoxycetanol, polyoxyethylene sorbitol monooleate, and polyoxyethylene sorbitan monooleate, respectively). Known emulsifying agents include, but are not limited to, lecithin and acacia. Known preservatives include, but are not limited to, methyl, ethyl, or n-propyl-para-hydroxybenzoates, ascorbic acid, and sorbic acid. Known sweetening agents include, for example, glycerol, propylene glycol, sorbitol, sucrose, and saccharin. Known thickening agents for oily suspensions include, for example, beeswax, hard paraffin, and cetyl alcohol.

Liquid solutions of the active ingredient in aqueous or oily solvents may be prepared in substantially the same manner as liquid suspensions, the primary difference being that the active ingredient is dissolved, rather than suspended in the solvent. Liquid solutions of the pharmaceutical composition of the invention may comprise each of the components described with regard to liquid suspensions, it being understood that suspending agents will not necessarily aid dissolution of the active ingredient in the solvent. Aqueous solvents include, for example, water and isotonic saline. Oily solvents include, for example, almond oil, oily esters, ethyl alcohol, vegetable oils such as arachis, olive, sesame, or coconut oil, fractionated vegetable oils, and mineral oils such as liquid paraffin.

Powdered and granular formulations of a pharmaceutical preparation of the invention may be prepared using known methods. Such formulations may be administered directly to a subject, used, for example, to form tablets, to fill capsules, or to prepare an aqueous or oily suspension or solution by addition of an aqueous or oily vehicle thereto. Each of these formulations may further comprise one or more of dispersing or wetting agent, a suspending agent, and a preservative. Additional excipients, such as fillers and sweetening, flavoring, or coloring agents, may also be included in these formulations.

A pharmaceutical composition of the invention may also be prepared, packaged, or sold in the form of oil-in-water emulsion or a water-in-oil emulsion. The oily phase may be a vegetable oil such as olive or arachis oil, a mineral oil such as liquid paraffin, or a combination of these. Such compositions may further comprise one or more emulsifying agents such as naturally occurring gums such as gum acacia or gum tragacanth, naturally-occurring phosphatides such as soybean or lecithin phosphatide, esters or partial esters derived from combinations of fatty acids and hexitol anhydrides such as sorbitan monooleate, and condensation products of such partial esters with ethylene oxide such as polyoxyethylene sorbitan monooleate. These emulsions may also contain additional ingredients including, for example, sweetening or flavoring agents.

A pharmaceutical composition of the invention may be prepared, packaged, or sold in a formulation suitable for rectal administration. Such a composition may be in the form of, for example, a suppository, a retention enema preparation, and a solution for rectal or colonic irrigation.

Suppository formulations may be made by combining the active ingredient with a non-irritating pharmaceutically acceptable excipient which is solid at ordinary room temperature (i.e. about 20° C.) and which is liquid at the rectal temperature of the subject (i.e. about 37° C. in a healthy human). Suitable pharmaceutically acceptable excipients include, but are not limited to, cocoa butter, polyethylene glycols, and various glycerides. Suppository formulations may further comprise various additional ingredients including, but not limited to, antioxidants and preservatives.

Retention enema preparations or solutions for rectal or colonic irrigation may be made by combining the active ingredient with a pharmaceutically acceptable liquid carrier. As is well known in the art, enema preparations may be administered using, and may be packaged within, a delivery device adapted to the rectal anatomy of the subject. Enema preparations may further comprise various additional ingredients including, but not limited to, antioxidants and preservatives.

A pharmaceutical composition of the invention may be prepared, packaged, or sold in a formulation suitable for vaginal administration. Such a composition may be in the form of, for example, a suppository, an impregnated or coated vaginally-insertable material such as a tampon, a douche preparation, or gel or cream or a solution for vaginal irrigation.

Methods for impregnating or coating a material with a chemical composition are known in the art, and include, but are not limited to methods of depositing or binding a chemical composition onto a surface, methods of incorporating a chemical composition into the structure of a material during the synthesis of the material (i.e. such as with a physiologically degradable material), and methods of absorbing an aqueous or oily solution or suspension into an absorbent material, with or without subsequent drying.

Douche preparations or solutions for vaginal irrigation may be made by combining the active ingredient with a pharmaceutically acceptable liquid carrier. As is well known in the art, douche preparations may be administered using, and may be packaged within, a delivery device adapted to the vaginal anatomy of the subject. Douche preparations may further comprise various additional ingredients including, but not limited to, antioxidants, antibiotics, antifungal agents, and preservatives.

As used herein, “parenteral administration” of a pharmaceutical composition includes any route of administration characterized by physical breaching of a tissue of a subject and administration of the pharmaceutical composition through the breach in the tissue. Parenteral administration thus includes, but is not limited to, administration of a pharmaceutical composition by injection of the composition, by application of the composition through a surgical incision, by application of the composition through a tissue-penetrating non-surgical wound, and the like. In particular, parenteral administration is contemplated to include, but is not limited to, subcutaneous, intraperitoneal, intramuscular, intrasternal injection, and kidney dialytic infusion techniques.

Formulations of a pharmaceutical composition suitable for parenteral administration comprise the active ingredient combined with a pharmaceutically acceptable carrier, such as sterile water or sterile isotonic saline. Such formulations may be prepared, packaged, or sold in a form suitable for bolus administration or for continuous administration. Injectable formulations may be prepared, packaged, or sold in unit dosage form, such as in ampules or in multi-dose containers containing a preservative. Formulations for parenteral administration include, but are not limited to, suspensions, solutions, emulsions in oily or aqueous vehicles, pastes, and implantable sustained-release or biodegradable formulations. Such formulations may further comprise one or more additional ingredients including, but not limited to, suspending, stabilizing, or dispersing agents. In one embodiment of a formulation for parenteral administration, the active ingredient is provided in dry (i.e. powder or granular) form for reconstitution with a suitable vehicle (e.g. sterile pyrogen-free water) prior to parenteral administration of the reconstituted composition.

The pharmaceutical compositions may be prepared, packaged, or sold in the form of a sterile injectable aqueous or oily suspension or solution. This suspension or solution may be formulated according to the known art, and may comprise, in addition to the active ingredient, additional ingredients such as the dispersing agents, wetting agents, or suspending agents described herein. Such sterile injectable formulations may be prepared using a non-toxic parenterally-acceptable diluent or solvent, such as water or 1,3-butane diol, for example. Other acceptable diluents and solvents include, but are not limited to, Ringer's solution, isotonic sodium chloride solution, and fixed oils such as synthetic mono- or di-glycerides. Other parentally-administrable formulations which are useful include those which comprise the active ingredient in microcrystalline form, in a liposomal preparation, or as a component of a biodegradable polymer systems. Compositions for sustained release or implantation may comprise pharmaceutically acceptable polymeric or hydrophobic materials such as an emulsion, an ion exchange resin, a sparingly soluble polymer, or a sparingly soluble salt.

Formulations suitable for topical administration include, but are not limited to, liquid or semi-liquid preparations such as liniments, lotions, oil-in-water or water-in-oil emulsions such as creams, ointments or pastes, and solutions or suspensions. Topically-administrable formulations may, for example, comprise from about 1% to about 10% (w/w) active ingredient, although the concentration of the active ingredient may be as high as the solubility limit of the active ingredient in the solvent. Formulations for topical administration may further comprise one or more of the additional ingredients described herein.

A pharmaceutical composition of the invention may be prepared, packaged, or sold in a formulation suitable for pulmonary administration via the buccal cavity. Such a formulation may comprise dry particles which comprise the active ingredient and which have a diameter in the range from about 0.5 to about 7 nanometers, and preferably from about 1 to about 6 nanometers. Such compositions are conveniently in the form of dry powders for administration using a device comprising a dry powder reservoir to which a stream of propellant may be directed to disperse the powder or using a self-propelling solvent/powder-dispensing container such as a device comprising the active ingredient dissolved or suspended in a low-boiling propellant in a sealed container. Preferably, such powders comprise particles wherein at least 98% of the particles by weight have a diameter greater than 0.5 nanometers and at least 95% of the particles by number have a diameter less than 7 nanometers. More preferably, at least 95% of the particles by weight have a diameter greater than 1 nanometer and at least 90% of the particles by number have a diameter less than 6 nanometers. Dry powder compositions preferably include a solid fine powder diluent such as sugar and are conveniently provided in a unit dose form.

Low boiling propellants generally include liquid propellants having a boiling point of below 65° F. at atmospheric pressure. Generally the propellant may constitute 50 to 99.9% (w/w) of the composition, and the active ingredient may constitute 0.1 to 20% (w/w) of the composition. The propellant may further comprise additional ingredients such as a liquid non-ionic or solid anionic surfactant or a solid diluent (preferably having a particle size of the same order as particles comprising the active ingredient).

Pharmaceutical compositions of the invention formulated for pulmonary delivery may also provide the active ingredient in the form of droplets of a solution or suspension. Such formulations may be prepared, packaged, or sold as aqueous or dilute alcoholic solutions or suspensions, optionally sterile, comprising the active ingredient, and may conveniently be administered using any nebulization or atomization device. Such formulations may further comprise one or more additional ingredients including, but not limited to, a flavoring agent such as saccharin sodium, a volatile oil, a buffering agent, a surface active agent, or a preservative such as methylhydroxybenzoate. The droplets provided by this route of administration preferably have an average diameter in the range from about 0.1 to about 200 nanometers.

The formulations described herein as being useful for pulmonary delivery are also useful for intranasal delivery of a pharmaceutical composition of the invention.

Another formulation suitable for intranasal administration is a coarse powder comprising the active ingredient and having an average particle from about 0.2 to 500 micrometers. Such a formulation is administered in the manner in which snuff is taken i.e. by rapid inhalation through the nasal passage from a container of the powder held close to the nares.

Formulations suitable for nasal administration may, for example, comprise from about as little as 0.1% (w/w) and as much as 100% (w/w) of the active ingredient, and may further comprise one or more of the additional ingredients described herein.

A pharmaceutical composition of the invention may be prepared, packaged, or sold in a formulation suitable for buccal administration. Such formulations may, for example, be in the form of tablets or lozenges made using conventional methods, and may, for example, 0.1 to 20% (w/w) active ingredient, the balance comprising an orally dissolvable or degradable composition and, optionally, one or more of the additional ingredients described herein. Alternately, formulations suitable for buccal administration may comprise a powder or an aerosolized or atomized solution or suspension comprising the active ingredient. Such powdered, aerosolized, or aerosolized formulations, when dispersed, preferably have an average particle or droplet size in the range from about 0.1 to about 200 nanometers, and may further comprise one or more of the additional ingredients described herein.

A pharmaceutical composition of the invention may be prepared, packaged, or sold in a formulation suitable for ophthalmic administration. Such formulations may, for example, be in the form of eye drops including, for example, a 0.1-1.0% (w/w) solution or suspension of the active ingredient in an aqueous or oily liquid carrier. Such drops may further comprise buffering agents, salts, or one or more other of the additional ingredients described herein. Other opthalmically-administrable formulations which are useful include those which comprise the active ingredient in microcrystalline form or in a liposomal preparation.

As used herein, “additional ingredients” include, but are not limited to, one or more of the following: excipients; surface active agents; dispersing agents; inert diluents; granulating and disintegrating agents; binding agents; lubricating agents; sweetening agents; flavoring agents; coloring agents; preservatives; physiologically degradable compositions such as gelatin; aqueous vehicles and solvents; oily vehicles and solvents; suspending agents; dispersing or wetting agents; emulsifying agents, demulcents; buffers; salts; thickening agents; fillers; emulsifying agents; antioxidants; antibiotics; antifungal agents; stabilizing agents; and pharmaceutically acceptable polymeric or hydrophobic materials. Other “additional ingredients” which may be included in the pharmaceutical compositions of the invention are known in the art and described, for example in Genaro, ed., 1985, Remington's Pharmaceutical Sciences, Mack Publishing Co., Easton, Pa., which is incorporated herein by reference.

Typically dosages of the compound of the invention which may be administered to an animal, preferably a human, range in amount from 1 μg to about 100 g per kilogram of body weight of the animal. While the precise dosage administered will vary depending upon any number of factors, including but not limited to, the type of animal and type of disease state being treated, the age of the animal and the route of administration. Preferably, the dosage of the compound will vary from about 1 mg to about 10 g per kilogram of body weight of the animal. More preferably, the dosage will vary from about 10 mg to about 1 g per kilogram of body weight of the animal.

The compound may be administered to an animal as frequently as several times daily, or it may be administered less frequently, such as once a day, once a week, once every two weeks, once a month, or even less frequently, such as once every several months or even once a year or less. The frequency of the dose will be readily apparent to the skilled artisan and will depend upon any number of factors, such as, but not limited to, the type and severity of the disease being treated, the type and age of the animal, etc.

B. A Method of Identifying a Useful Compound

The invention encompasses a method for identifying a compound that inhibits PRCP. One skilled in the art would appreciate, based upon the disclosure provided herein, that assessing the expression, activity, or both, of PRCP can be performed by assessing, inter alia, the levels of PRCP in an animal, and the like, when compared to the same parameter in an otherwise identical animal not treated with the compound. One skilled in the art would understand that such compounds can be useful for, decreasing food intake, decreasing percent body fat, increasing the resting metabolic activity, and initiating weight loss. This is because the data disclosed elsewhere herein demonstrate, for the first time, that inhibiting PRCP (expression, activity, or both) mediates a variety of beneficial effects, including, but not limited to, decreased food intake, decreased body fat, weight loss, decreased caloric intake, increased resting metabolic activity, and initiating weight loss. This is because the present invention discloses, for the first time, that PRCP expression is associated with, or mediates, such diseases, conditions and disorders. Accordingly, the data disclosed elsewhere suggest that inhibiting of PRCP can provides a useful therapeutic for those diseases, disorders or conditions.

The method comprises administering to a mammal a compound and comparing the level of PRCP activity in the mammal before and after administration of the compound. The routineer would understand, based on the disclosure provided herein, that a lower level of PRCP activity in the mammal after administration of the compound compared with the level of PRCP activity before administration of the compound indicates that the compound is useful for treating a disorder, condition or disease related to obesity, including, but not limited to, hypertension, dyslipidemia, diabetes, stroke, gallbladder disease, cardiovascular disease, osteoarthritis, rheumatoid arthritis, hypercholesterolemia, stable angina, unstable angina, sleep apnea, respiratory problems, cancer, stroke hyerinsulinemia, Syndrome X, hypercholesterolemia, hyperlipoproteinemia, hypertriglyceridemia, atherosclerosis, diabetic renal disease, and the like.

This is because, as stated previously elsewhere herein, it has been discovered that inhibiting PRCP activity in an animal treats a disease associated with PRCP expression and/or activity. The skilled artisan would also appreciate, in view of the disclosure provided herein, that assays to determine the level of PRCP activity and/or expression in a mammal include those well known in the art, or those to be developed in the future, all of which can be used to assess the level of PRCP activity in a mammal before and after administration of the compound. The skilled artisan would further appreciate that PRCP activity, as disclosed elsewhere herein, includes an association with resting metabolism rate, caloric consumption, and the like. Further, the invention encompasses a compound identified using this method.

The invention further includes another method of identifying a compound that useful for treating obesity. The method comprises assessing the level of PRCP activity in a mammal after administration of a compound compared to a standard baseline previously established for that particular species of mammal before administration of the compound. Methods of assaying PRCP activity are disclosed elsewhere herein, but can include assessing caloric intake, resting metabolic activity, amount of food intake, and weight loss. A decrease in PRCP activity in a mammal after administration of a compound compared to the baseline level of PRCP activity determined for the that particular species of mammal before administration of a compound can indicate that a compound is useful in inhibiting the activity of PRCP, and is therefore useful in the treatment of obesity. This is because, as discussed previously elsewhere herein, inhibition of PRCP mediates a decrease in, inter alia, food intake and weight, thereby treating obesity, such that it would be appreciated by the skilled artisan, based on the disclosure provided herein, that a compound that inhibits PRCP can be used to treat obesity.

The invention encompasses a method of identifying a compound that is useful for treating obesity. The method comprises assessing the level of PRCP activity in a mammal after administration of a compound compared to the level of PRCP in the same mammal prior to the administration of the compound, or to the level of PRCP activity in an otherwise identical mammal to which the compound is not administered. A decrease in PRCP activity in a mammal after administration of a compound compared to the level of PRCP activity determined for the mammal prior to the administration of the compound, or compared with the level of PRCP activity in the otherwise identical mammal to which the compound is not administered, is an indication that the compound is useful for the treatment of obesity. This is because, as more fully discussed elsewhere herein, the data disclosed herein demonstrate, for the first time, that inhibiting the activity of PRCP mediates a decrease in, inter alia, food intake and weight, thereby treating obesity, such that it would be appreciated by the skilled artisan, based on the disclosure provided herein, that a compound that inhibits PRCP can be used to treat obesity.

The invention encompasses a method of identifying a compound that is useful for treating obesity. The method comprises assessing the level of PRCP activity in a cell after the cell is contacted with a compound compared with the level of PRCP in an otherwise identical cell which is not contacted with the compound. A decrease in PRCP activity in the cell contacted with the compound compared with the level of PRCP activity in the otherwise identical cell not contacted with the compound is an indication that the compound is useful for the treatment of obesity. This is because, as more fully discussed elsewhere herein, the data disclosed herein demonstrate, for the first time, that inhibiting the activity of PRCP mediates a decrease in, inter alia, food intake and weight, thereby treating obesity, such that it would be appreciated by the skilled artisan, based on the disclosure provided herein, that a compound that inhibits PRCP can be used to treat obesity.

The skilled artisan would appreciate, based upon the disclosure provided herein, that the cell used to assess the level of PRCP activity due to the cell being contacted with a compound can be selected from the group consisting of a liver cell, a brain cell, a gonadal white adipose cell, a gastrocnemius muscle cell, and the like.

The skilled artisan would appreciate, based upon the disclosure provided herein, that for each of the methods of identifying a compound of interest, the invention encompasses any compound identified thereby.

### II. Transgenic Animals

The skilled artisan will appreciate, as disclosed elsewhere herein, that a transgenic animal comprising a deficiency in PRCP is useful in the study of obesity, obesity-related disorders, and the like.

The skilled artisan will also appreciate, when armed with the present disclosure, that a transgenic animal comprising a deficiency in PRCP is useful in that the transgenic mammal will comprise a lower amount of body fat, and is therefore a more healthful animal for human consumption. That is because, as disclosed herein, a transgenic animal comprising a deficiency in PRCP is of a similar size to a counterpart animal comprising a functional PRCP, but the transgenic animal comprises less body fat, and is therefore a leaner animal. Further, it would be understood by the routineer, based upon the disclosure provided herein, that a reduction in consumption in animal fat is widely regarded as a method to not only reduce obesity, but to maintain and improve cardiovascular health and to prevent and/or treat diabetes.

The present invention encompasses a non-human transgenic animal which is commercially relevant as a food stuff for human consumption, as well as animals not used as food stuffs, but where a leaner animal is of some benefit either to the animal or to humans. Such animals include, but are not limited to, a bird, a rodent, a cow, a pig, a sheep, a buffalo, a beefalo, a bison, a deer, a goat, and the like.

In addition, the skilled artisan would appreciate, based upon the disclosure provided herein, that a transgenic non-human mammal lacking PRCP function is a useful model system for the study of a disease or condition associated with, or mediated by, PRCP function, including a condition where lack of PRCP provides a benefit to the mammal. Thus, the transgenic non-human mammal of the invention is not only useful as a leaner comestible, but also provides a useful model system for studying the role of PRCP function, especially as it relates to obesity, diabetes, food intake, and body weight control.

The present invention therefore includes a transgenic non-human animal comprising a deficiency in PRCP. The skilled artisan will appreciate, when equipped with this disclosure and the data contained herein, that an animal deficient in PRCP comprises an animal that lacks the PRCP gene in its entirety, or any portion thereof, and is not limited to the portion exemplified herein.

Further, one of skill in the art will understand, when armed with the present disclosure, that PRCP deficiency encompasses no expression, insufficient and/or decreased expression, and/or the production of a non-functional PRCP, in that it does not exhibit the activity of PRCP as disclosed herein.

The skilled artisan, once equipped with the teachings disclosed elsewhere herein, will readily appreciate how to produce transgenic animals deficient in PRCP. The skilled artisan will also appreciate, while recognizing that the disclosure contained herein is in no way limiting to the methods to produce a transgenic animal deficient in PRCP, that such animals can be produced by the deletion of the entire gene, or portions thereof. Further, the skilled artisan will appreciate that a transgenic animal deficient in PRCP can be produced by introducing nonsense, missense, or other mutations in the coding regions of PRCP gene. Further, one of skill in the art will appreciate that such animals can be generated by mutations in the promoter/enhancer region of the regulatory elements governing PRCP expression. As disclosed elsewhere herein, methods to effect the expression of PRCP can also encompass expression of an oligonucleotide antisense to the PRCP gene, or some portion thereof, as well as the use of ribozymes and synthetic antibodies for the generation of a transgenic animal comprising a deficiency in PRCP. All such embodiments disclosed herein are encompassed in the present invention.

### EXPERIMENTAL EXAMPLES

The invention is now described with reference to the following Examples. These Examples are provided for the purpose of illustration only and the invention should in no way be construed as being limited to these Examples, but rather should be construed to encompass any and all variations which become evident as a result of the teaching provided herein.

For the following experiments we used NCBI Accession No. NP_{—}082519 (GI 33469015), a 491 aa encoding Mus musculus angiotensinase C like protein and NCBI Accession No. AAH55022 (GI:32967631), a 491 aa encoding Mus musculus PRCP protein. See, also Strausberg et al., Proc. Natl. Acad. Sci. U.S.A. 99(26): 16899-16903 (2002).

Obesity is a complex trait influenced by diet, energy expenditure and genetics. Identification of genes that underlie human obesity has been difficult, but mouse models have been used to clone and identify genes subsequently shown to contribute to human obesity. Mice provide a model to find naturally occurring mutations that cause obesity. Mice are the best available model, other than humans, for genetic studies of obesity because of their well-defined functional homology with the human genome, because obesity is influenced by hormones and neural signals from many tissues that are shared with all mammals but which are not all present in non-mammalian models, and because of our ability to alter their environment using diets that humans could eat. Further, positional cloning can identify genes that influence phenotypes, because the genes map coincident with the trait. Homologues and alleles of these genes can then be tested for their effects on human physiology. Congenic models such as those used here have been used to dissect the genetics of complex traits.

### Example 1

### Identification of PRCP Gene from a Congenic Mouse Line

Material and Methods

Expression Array Analysis

Applied Biosystems Mouse Genome Survey Arrays were used to analyze the transcriptional profiles of twenty-four RNA samples (two mice strains, three mice per tissue and four tissues-brain, gonadal white adipose, liver, and gastrocnemius) in this study. The Applied Biosystems Mouse Genome Survey Array contains 32,996 60-mer oligonucleotides probes representing a set of 32,381 individual mouse genes and more than 1,000 control probes.

Digoxigenin-UTP labeled cRNA was generated and linearly amplified from 1 μg of total RNA using Applied Biosystems Chemiluminescent RT-IVT Labeling Kit v 2.0 and manufacturer's protocol. Array hybridization, Chemiluminescence detection, image acquisition and analysis were performed using Applied Biosystems Chemiluminescence Detection Kit and Applied Biosystems 1700 Chemiluminescent Microarray Analyzer following manufacturer's protocol. Briefly, each microarray was first pre-hybridized at 55° C. for 1 hr in hybridization buffer with blocking reagent. 18 μg of labeled cRNA targets were first fragmented by incubating with fragmentation buffer at 60° C. for 30 min, mixed with internal control target (ICT, 24-mer oligo labeled with LIZ fluorescent dye) and hybridized to each pre-hybed microarray in a 1.5-ml volume at 55° C. for 16 hr. After hybridization, the arrays were washed with hybridization wash buffer and chemiluminescence rinse buffer. Enhanced chemiluminescent signals were generated by first incubating arrays with anti-digoxigenin-Alkaline Phosphatase, enhanced with Chemiluminescence Enhancing Solution and finally adding Chemiluminescence Substrate. Images were collected for each microarray using the 1700 analyzer. Images were auto-gridded and the chemiluminescent signals were quantified, corrected for background and spot and spatially normalized.

Statistical Analysis

For each experiment, means were compared between experimental groups using one-way analysis of variance (ANOVA) with mean comparisons by the Student-Newman-Keuls Method. A level of confidence of p<0.05 was used to determine significant differences.

Real Time PCR to Quantitate PRCP mRNA in Brain

RNA was isolated from male littermate mice derived from a cross of F1 B6x B6.C-D7Mit353 mice. Whole brains were removed from mice after an overnight fast and assays performed by real time PCR in an ABI 7900. Genotypes are: “B” for background (n=8), “H” for heterozygote (n=9), and “C” for congenic or BALB/c (n=7). The assays used target three separate exons: TaqMan assay 814504 targets exon 3, TaqMan assay 814505 targets exon 5, and TaqMan assay 1324043 targets exon 2. In addition a separate assay was performed using custom primers that were detected with Sybr green. Five separate control genes were used: one with Sybr Green (36B4) and four with TaqMan. Genotype effects (deltas) were determine for all primers (Prcp and control genes). Values for the control genes were averaged. The exon 2 primers were calculated using a separate control gene assay for beta-glucuronidase since this was from a separate cDNA synthesis of the same RNA. Overall fold increased expression was calculated as described by ABI. Results are shown in Table 5 and indicate a 1.7 fold higher expression of Prcp mRNA in lean congenic than more obese C57BL/6J mice.

Results

Recently, a B6.C-D7Mit353 subcongenic mouse line with less body fat than C57BL/6J background controls has been identified (Diament et al., 2003). It is believed that the phenotypic differences (such as obesity) between background and congenic strains are due to polymorphisms in genes located in the congenic donor region located on chromosome 7. The lean subcongenic line contains the D7Mit353 BALB/c allele (congenic line name B6.C-D7Mit353) on chromosome 7 and donor strain DNA from the BALB/c strain surrounded by B6 genomic DNA for the rest of the genome. The maximum extent of the BALB/c donor region is between D7Mit37 to D7Mit96 that has the B6 alleles in the congenic. This donor region includes 97 genes that may have BALC/c alleles. (Table 3).

In order to identify these genes, a whole genome oligonucleotide based mouse microarray (ABI1700) was used to search for differentially expressed genes in biological triplicates from mice homozygous for the background “B” and congenic “C” genotypes. mRNA expression from liver, brain, gonadal white adipose tissue and gastrocnemius muscle were examined since obesity may be influenced by gene expression in many tissues. RT-IVT amplified samples were hybridized to one of 29 separate microarrays, including duplicate hybridizations for 5 of 6 brain samples. Chemiluminescent signals were normalized across all arrays using quantile normalization method. Normalized data were analyzed using the Local Pooled Error (LPE) test of Jain et al. to identify differentially expressed genes. For each tissue, only genes that had signal/noise ratio (S/N)>=3 (i.e. 99.9% probability of the signal being above the background) on 2 out of 3 (4 for brain) arrays within either mouse strain were included in the statistical analysis. Raw p-values were then adjusted for multiple testing by calculating false discovery rates (FDR).

These analysis identified 330 genes passing FDR=0.05 and 188 genes passing FDR=0.01 in any one of the four examined tissues. None of these genes was in the congenic donor region. Seven genes in the congenic donor region are differential in one of the tissues with raw p values <0.01, including three candidate obesity genes. Dgat2 is known to influence triglyceride synthesis. There is a suggestive genotype effect in muscle (see Table 1 and Table 3 where all 97 genes were mapped to the congenic region). However, Dgat2 is unlikely to be responsible for the observed congenic phenotype both because it does not exhibit statistically significant regulation and because its levels are regulated opposite to those expected based on studies of Dgat2 knockout mice. Dgat2 mRNA levels are lower in the obese background strain mice, while mice with knockouts for Dgat2 have no triglyceride (Table 1). Thyroid hormone responsive spot 14 (Thrsp) is included among the four most significant genes from the congenic donor region from brain. Real time PCR for Thrsp mRNA in liver demonstrated a genotype effect that is consistent to the liver microarray data, but which was not statistically significant. The array probe for the Mgat2 (monoacylglycerol 0-acyltransferase 2) gene, that is immediately next to Dgat2, did not have detectable signals on more than 1 array for any tissues-strain.

For this reason, the focus of the subsequent studies were shifted to gene prolylcarboxypeptidase (Prcp, also known as angiotensinase C or Pro-x carboxypeptidase). This is because gene Prcp has previously been associated with the metabolic syndrome of humans (McCarthy et al., 2003) and because it is functionally related to carboxypeptidase E (Cpe) that is the underlying cause of the “fat” mutation in mice. Tests by real time PCR were done using four separate primers, targeting exon 2, 4 and 5, and five separate endogenous control genes to rule out artefacts due to unexpected splice variants and genotype effects on control genes. These results demonstrated statistically significant 1.77±0.33 (SD) fold higher expression of Prcp in brains of background mice than congenic mice, 1.48±0.31 fold higher expression in heterozygous than congenic mice and 1.19±0.049 fold higher expression in background than heterozygotes mice. These results are consistent with an additive model with the congenic allele promoting increased PRCP expression. To search for functional differences between background and congenic alleles of Prcp, both the donor and background alleles of Prcp were sequenced. No coding, 3′ or 5′ untranslated region variants were found. One promoter base change, a C→T on the donor strain was found −718 bases upstream of the start codon. Mouse Prcp is orthologous to human prolylcarboxypeptidase (Prcp). Prcp in mouse is located at 81.3 Mb on chromosome 7 (Diament et al., 2003). The human ortholog is located at 82.3 Mb on chromosome 11.

### Example 2

### α-MSH as a Substrate of PRCP

Material and Methods

Materials

α-MSH (Ac-STSMEHFRWGKPV-NH₂) (SEQ ID NO: 30) and angiotensinogen II (AgII) were purchased from Phoenix Pharmaceuticals. Inc, (Belmont, Calif.). MSH₁₋₁₂ (Ac-STSMEHFRWGKP-COOH) (SEQ ID NO: 31) was synthesized in its purified form by the W. M. Keck Foundation Biotechnology Resource Laboratory at Yale University. HD-Pro-Phe-Arg-paranitroanilide (S2302) was from DiaPharma (Franklin, Ohio). Human high molecular weight kininogen (HK) (18 Units/mg) and prekallikrein (PK) (21 Units/mg) was obtained from Research Enzyme Laboratory (South Bend, Ind.). pMT/BiP/V5-His C was obtained from Invitrogen Laboratories (Carlsbad, Calif.). N-benzyloxycarbonyl-prolyl-prolinal (Z-PP) was purchased Sigma. B-PP was a kind gift of Dr. Sherwin Wilk, Mount Sinai School of Medicine, New York.

Cloning and Expression of PRCP

pMT/BiP/V5-His C was employed as the basal plasmid in vector construction. Human PRCP cDNA, lacking the first 391 base pairs of PRCP (Prcpₙₜ₃₉₁₋₁₈₄₅), was subcloned into the vector at the Kpn1 and EcoR1 multiple cloning site forming pMT/Bip/V5-HisC-PRCP. The construction methods of the PRCP expression vector and the recombinant PRCP expression in Schneider 2 cells followed the standard protocols (Shariat-Madar et al., 2002). The PRCP has a predicted molecular mass of 51 kDa (rPRCP₅₁)

### Substrate Inhibition Assay

Samples containing 8 nM rPRCP₅₁ in 100 ml total volume were prepared in the absence or presence of increasing concentration of peptide α-MSH, MSH₁₋₁₂ or AgII in HEPES carbonate buffer (137 mM NaCl, 3 mM KCl, 12 mM NaHCO₃, 14.7 mM HEPES, 5.5 mM dextrose and 0.1% gelatin, pH 7.1 containing 10 mM CaCl₂, and 1 mM MgCl₂) in microtiter plate cuvette wells with previously absorbed HK at 1 mg/well and with 20 nM PK at 37° C. The ability of purified rPRCP₅₁ to activate PK bound to HK on plastic microtiter plates was determined in the absence or presence of increasing concentrations (0.001-1 mM) of the peptide (McCarthy et al., 2003; Shariat-Madar et al., 2002). After incubation, the wells were washed to remove unbound rPRCP and peptide and any formed kallikrein activity was determined by addition of 100 μl of S2302 (0.8 mM) and hydrolysis was observed at 405 nm for 1 h at 37° C. The rate of hydrolysis was recorded at different concentrations of peptide and expressed as percent kallikrein activity remaining. Results were expressed as mean±SEM of 3 or more experiments.

Results

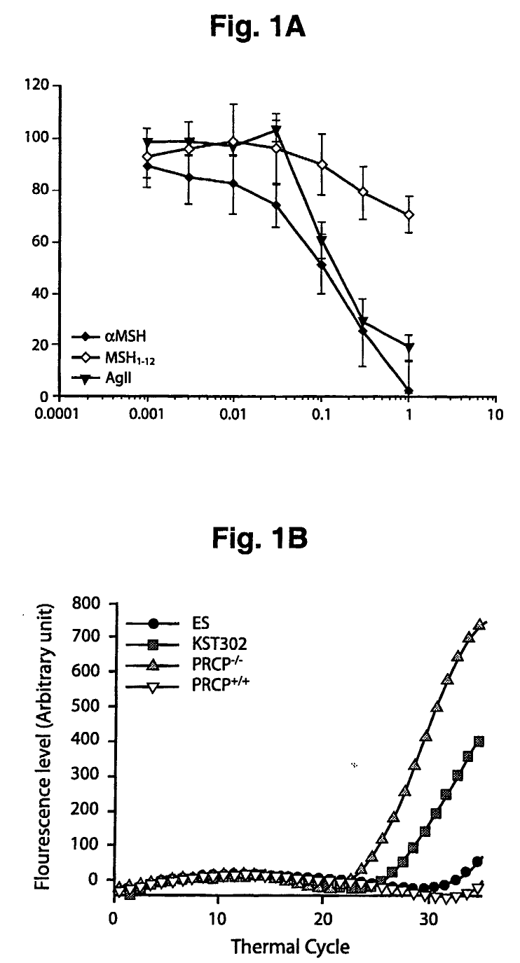

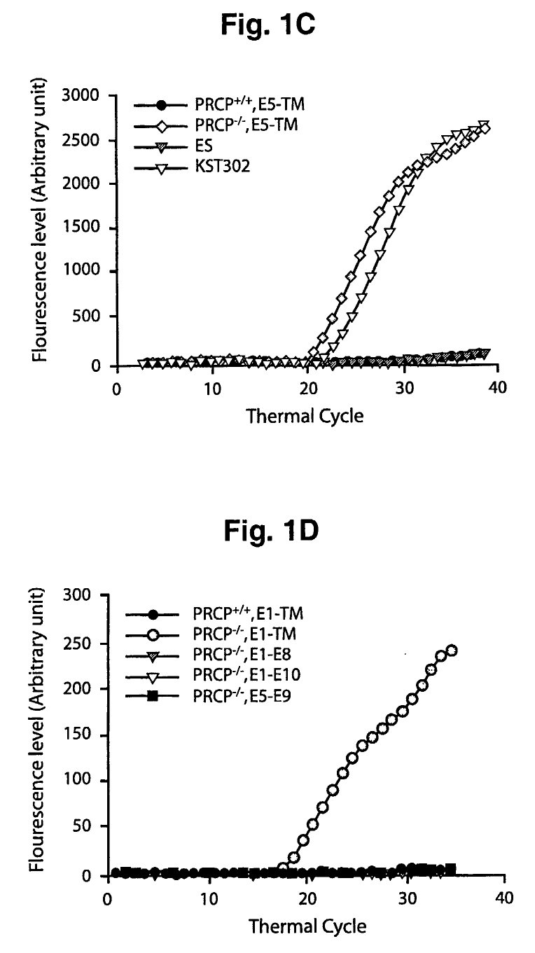

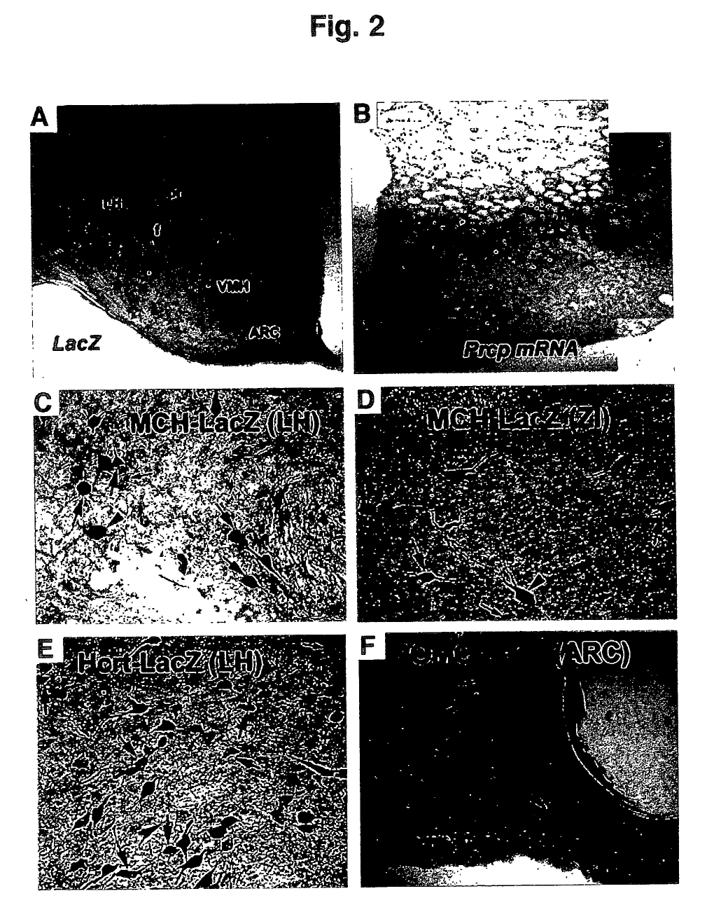

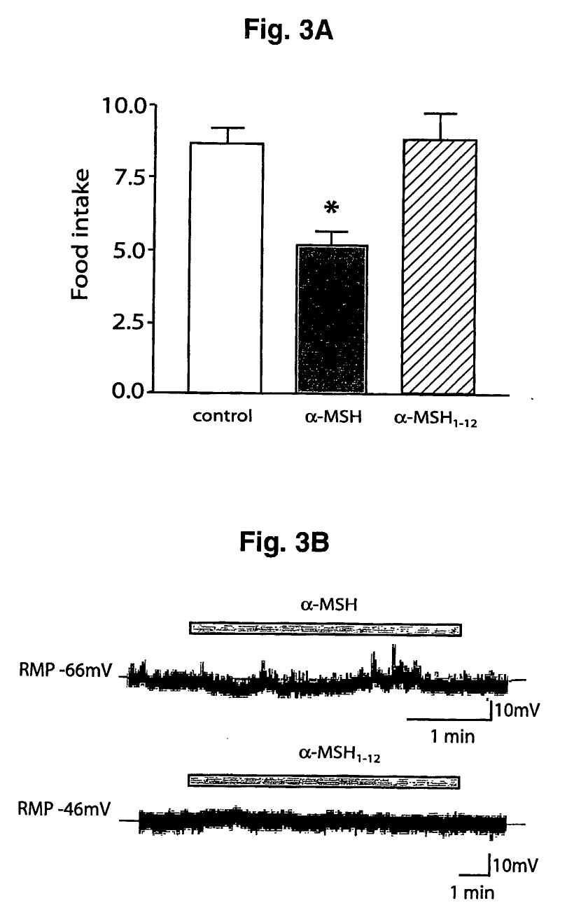

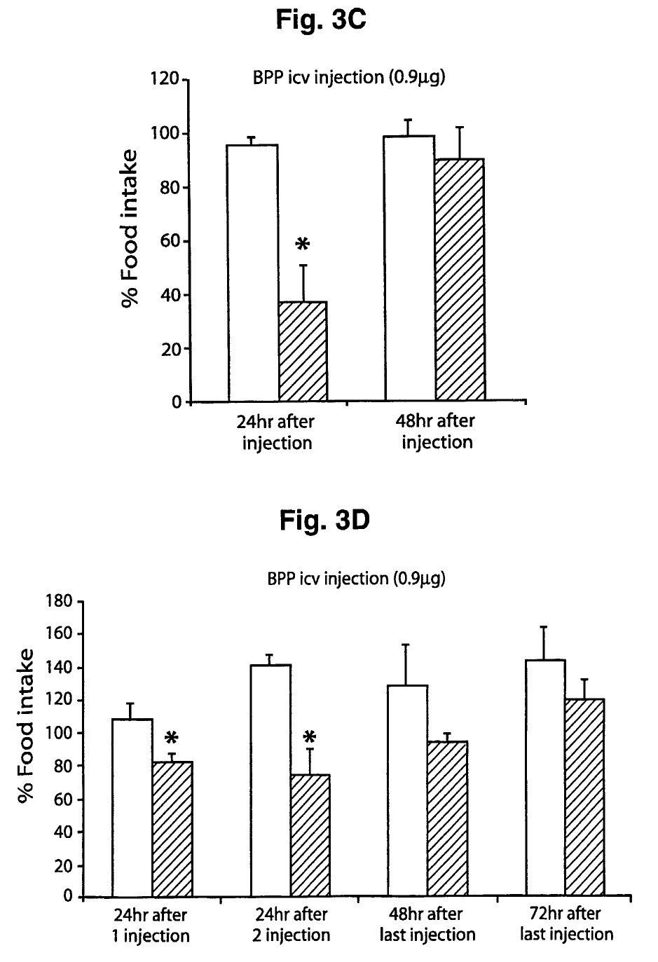

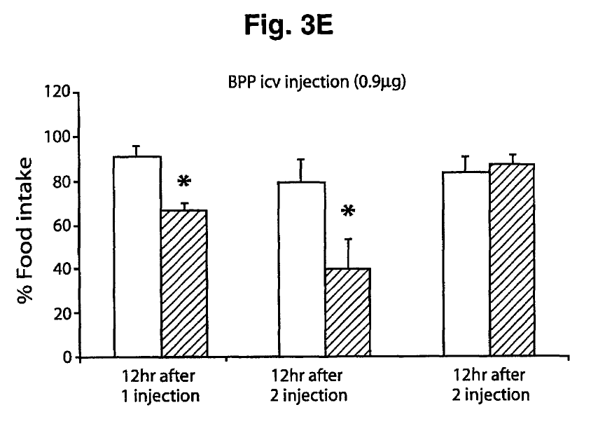

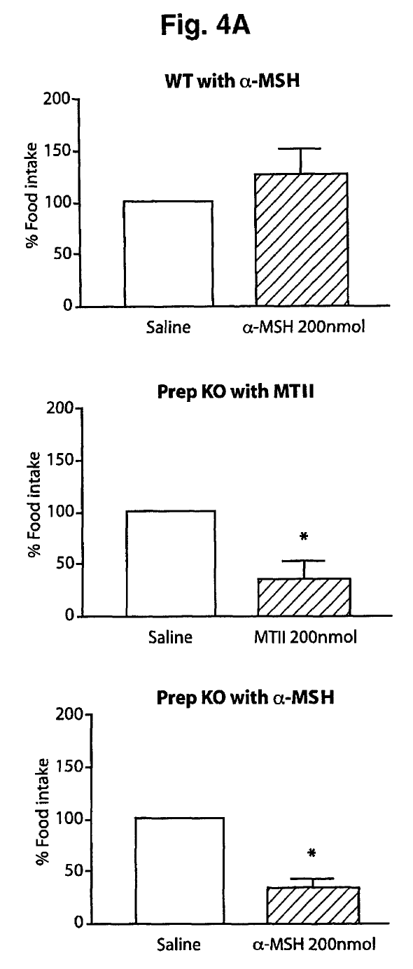

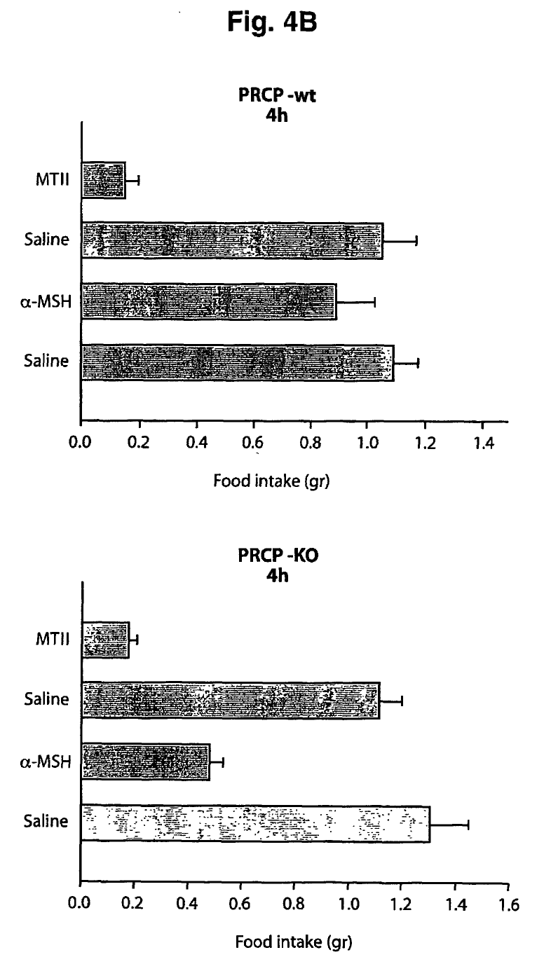

PRCP is a membrane bound protein that cleaves C-terminal amino acids linked to a penultimate proline and can function as a protease at physiologic pH. Thus, because of its amino acid sequence, α-MSH, a critical anorexigenic neuromodulator in the hypothalamus is a putative substrate of PRCP. To confirm, an in vitro assay was performed to determine if α-MSH is a substrate inhibitor of rPRCP₅₁-induced prekallikrein (PK) activation (FIG. 1A) (Shariat-Madar et al., 2002; 2004). Recombinant PRCP₅₁ activates PK with a Kₘ and Vₘₐₓ of 9 nM and 0.2 min⁻¹, respectively. Incubation of PRCP with α-MSH at 0.001-1 mM led to a gradual decrease in the activation of PK by rPRCP₅₁ with an IC₅₀=100 mM. Likewise, angiotensinogen II (AgII), an established substrate of PRCP, inhibited PK activation with an IC₅₀=150 mM, as was shown previously (Shariat-Madar et al., 2002; 2004; Odya et al., 1978). Alternatively, the putative product of PRCP hydrolysis of α-MSH, α-MSH₁₋₁₂, was much less effective in blocking rPRCP₅₁ activity, achieving about 20% inhibition at 1 mM (FIG. 1A). These data show that α-MSH₁₋₁₃ with a C-terminal Pro-Val bond, but not MSH-₁₋₁₂ that lacks the C-terminal Val, was a substrate of PRCP.

### Example 3

### Generation of PRCP Deficient Mice

Material and Methods

Animal Husbandry

Mice were housed and cared for according to Duhl et al. Briefly, once a subcongenic line was made, two heterozygous mice with the subcongenic region were mated, so that their progeny had one of three genotypes. This arrangement controls for all controllable environmental variance by using sibling mice as control mice. Mice were fed a low fat diet, weaned at 3 weeks, housed 5 per cage, and dissected at 16 weeks of age.

Preparation of PRCP Knockout Mice

ES cells (KST302) heterozygous for PRCP deletion using gene-trap vectors were generously provided by Dr. William Skarnes, University of California at Berkeley through BayGenomics [NHLBI-Bay Area Functional Genomics Consortium (http://baygenomics.ucsf.edu)] (Mountjoy et al., 1994). These PRCP gene trapped cells contain an insertion that has the following regions in its vector (pGT 1-8TM) from 5′ to 3′ called SA (splice acceptor), CD4-TM, and a lacZ reporter (Skarnes et al., 1995). The KST302 cells were microinjected into C57BL/6J blastocysts at the University of Michigan Transgenic Animal Model Core and surgically implanted in pseudopregnant female recipients in a 129 background. Germline transmission was indicated by the presence of agouti coat color. Since the cloning vector contains a lacZ reporter gene, mice deleted of PRCP were screened by real time PCR using primers for LacZ (bgal) (Table 4). Genotyping of the knockout mice was performed by PCR using DNA purified from tail biopsies with a spin column according to the manufacturer's specifications (Promega, Madison, Wis., USA). DNA from wild type embryonic stem (ES) and KST302 (PRCP^{+/-} clone) cells were used as control. The PCR conditions were 94° C. for 5 min (one cycle); 94° C. for 1 m, 60° C. for 1 m, 72° C. for 2 m (35 cycles); and 72° C. for 10 min. With each run of the real time PCR, the relative expression of PRCP gene was normalized to that of g-actin in order to insure fidelity between runs according to the procedure of Schmittgen et al. Animals with bgal expression on real time PCR similar to KST302 cells were considered heterozygous for PRCP deletion. Animals with bgal expression greatly increased over KST302 cells were considered homozygous. At the time of the present experiments, PRCP deleted mice had been backcrossed six generation into a C57BL/6 background before homozygous mice were prepared by breeding.

Total RNA Extraction

At time of mouse dissection, liver, spleen, brain, gastrocnemius, kidneys and four fat pads (femoral, epidydimal, retroperitoneal, and mesenteric) were removed and flash frozen in liquid nitrogen. These samples were subsequently stored at −80 until RNA extraction. Approximately 100 mg of sample tissue was used for RNA extraction using TRIzol (Life Technologies, Grand Island, N.Y.) according to the manufacturer's protocol.

Reverse Transcription

Single-stranded cDNA was synthesized using 1 μg of total RNA and TaqMan reverse transcription kit (Applied Biosystems, Foster City, Calif.) according to the manufacturer's protocol.

Sequencing

Sequencing was done on cDNA for all but the 5′ UTR and 3′UTR regions of the genes. To sequence the 5′UTR and 3′UTR, genomic DNA was used and primers designed based off of sequences from the Celera database. Primers were used to both amplify DNA, and later to sequence the PCR fragment after a gel extraction using QIAquick Gel Extraction Kit (Qiagen Inc., Valencia, Calif.). These primers are listed in Table 2. Primers were designed using the program Eugene v 2.2 (Daniben Inc., Cincinnati, Ohio).

Characterization of PRCP Knockout (KO) Mice

Real time quantitative (Taqman) PCR analysis was carried out as described by the vendor's instructions, with minor modifications. Briefly, measurements were performed using the iCycler iQ real-time PCR detection system (BioRad, Hercules, Calif.). The primers (Invitrogen) and probes (ITD, Coralville, Iowa) were designed. Probes were 56-labeled with 6-carboxyfluorescein (FAM) and a downstream 3 Black Hole quencher dye (BHQ-1) (Integrated DNA Technologies Coralville, Iowa) (Table 4). Specific primers for the real time PCR were used for the RT-PCR assay examining insertional mutation site in kidney PRCP RNA. Melting profiles showed the generation of specific products with melting temperatures of 57.3° C. The PCR mixture (50 ml) consisted of 0.2 mM of each primer, 0.02 mM probe, 1 mg RNA and TaqMan Universal Master Mix (2×) or platinum Taq. Reaction conditions were used: 95° C. for 5 min, followed by 35 cycles at 95° C. for 1 m and 58 or 60° C. for 1 m. Real-time PCR data were expressed as relative quantity based on the ratio of fluorescent change. Negative controls (samples without polymerase) were performed in parallel during different determinations to assess melting curve and assure equivalent assay conditions. cDNA products were also analyzed for purity by gel electrophoresis and sequencing. All assays were performed in triplicate and reported as the mean.

Sybr® Green Quantitative PCR

Prcp and control gene Arbp were also used to quantify RNA levels in tissue. The control gene Arbp is a 60S acidic ribosomal protein used to normalize loading and total RNA sample variations. The following are the Sybr® Green reagent and cDNA concentrations: 1.5 μl (30 ng) of cDNA from the RT reaction. 2.5 μl of 3 μM forward primer, 2.5 μl of 3 μM reverse primer, 12.5 μl of 2× Sybr Green PCR Mastermix (Applied Biosystems Inc., Warring UK), and 6.0 μl Ambion Nuclease free water to make a 25 μl reaction volume. All primers designed to be used with Sybr® Green technology were created with Primer Express v 2.0.0 (Applied Biosystems, Foster City, Calif.) and the sequences of these primers are found in Table 2. To determine cDNA contamination in reagents, a no template control (NTC) replaced the 1.5 μl of cDNA with water. No reverse transcriptase reactions (−RT) controls were run to check for genomic DNA contamination and amplification. For these reactions, 1.5 μl reverse transcriptase was replaced with 1.5 μl water. Ideally these controls should have Cₜ values at cycle 40 (see Data Analysis and Statistical Evaluation), indicating no amplification. Everything was run in triplicate. Pooling of (−RT) reactions were done in order to conserve reagents. No positive amplification of −RT reactions ever required us to doubt expression levels of +RT reactions. Each PCR plate was covered with an optical adhesive cover and inserted in ABI Prism 7900HT Sequence Detection System machine. The thermal cycling conditions were as follows: 50° C. for 2 minutes, 95° C. for 10 minutes, 40 cycles of 95° C. for 15 seconds and 60° C. for 1 min, followed by a dissociation curve using a gradual temperature ramping from 60° C. to 95° C. over a 5 minute interval with fluorescent measurements taken every 7 to 10 seconds. All reactions were performed using Model 7900HT Sequence Detector (PE Applied Biosciences, Foster City, Calif.).

Body Temperature

At approximately 15 weeks of age, anal temperature was taken. The animals were restrained by covering the entire mouse with a small box that had a slit cut out of the back of the box to allow only the tail outside of the box. An anal temperature probe was lubricated with KY Jelly and inserted approximately 1 cm into the rectum of the mouse. The probe was left in for approximately 10-15 seconds while the temperature stabilized to 0.1 degrees Celsius. This procedure was done at approximately 3 PM for three consecutive days and an average temperature was used for statistical analysis.

Running Wheel Activity

At approximately 8 weeks of age mice were individually housed in polycarbonate cages equipped with electronically monitored running wheels (Mini Mitter Co., Bend, Oreg.) cage with a running wheel and allowed free access to food and water. Running wheel data, expressed as total revolutions per day, were collected at 15 minutes intervals using the VitalView Data Acquisition System (Mini Mitter, Bend, Oreg.). Mice were adjusted to the new running wheel and room environment for 2 days before measurements were taken. Measurements were taken for five consecutive days and calculated as “average turns per day”.

Food Intake

At 7 weeks of age, mice were individually housed in wire mesh cages, and allowed free access to food and water. Mice were adjusted to the new cage and environment for 3 days before measurements were taken. Mice and the amount of food were weighed at the beginning of the week. At the end of seven days, the mice and food left in the cage were recorded. Spillage and feces were separated and the food spillage excluded from the food consumed. This procedure was repeated for the following week with the same mice. A one-week food intake measurement period was also done at 14 weeks of age. The materials and methods were the same as the 7-8 weeks of age.

Spontaneous Activity

Each mouse was tested for normal exploratory locomotion. The mouse was placed in the photocell-equipped automated open field box. Accuscan software calculated total distance traversed, horizontal activity and stereotypy. The room was maintained at 22±1° C. on a 12-h light/dark cycle. Locomotor activity was measured using a Digiscan Animal Activity Monitoring System (Omnitech Electronics, Columbus, Ohio). Mice were put in the Digiscan at 8 weeks of age with pine shavings bedding, and allowed free access to food and water. Mice were adjusted to the new room and environment for 3 days, and measurements averaged over 4 days.

Metabolic Rate

Mice were shipped to Baylor University for metabolic rate analysis. Mice were shipped at approximately 5 weeks of age and measured at approximately 16 weeks of age. Mice were single housed. Daytime and night time resting energy expenditure, O₂, CO₂, heat, and weight of the mouse were measured.

Data Analysis and Statistical Evaluation

Analyses of real-time quantitative PCR data were performed using the comparative threshold cycle (Cₜ) method as suggested by Applied Biosystems. Cₜ values are defined as the PCR amplification cycle in which the reporter signal is greater than the minimal detection level, and Cₜ is inversely related to the relative abundance of a particular transcript. Only samples with Cₜ values at least five units less than that of the “no-RT” control were included in data analysis. To correct for total RNA loading variations, the expression of the gene tested was measured relative to the expression of control gene of Abrp. Quantification of mRNA was measured in triplicate and the Ct values were averaged. The relative expression of gene tested compared to Abrp was calculated by the formula 2^{[Ct(36B4)-Ct(Gene Tested)]}. For ease of understanding and visual presentation, the values were converted to represent fold increase from the lowest expressed genotype such that the value of the lowest expressed genotype was given the arbitrary value of 1.0 and all other genotypes compared to that. This calculation was accomplished by dividing all relative expression values by the relative expression value of the lowest expressing genotype. Statview (SPSS Inc., Chicago, Ill.) was used for statistical analysis. To establish whether mRNA level differences were statistically significant for any group, one-way analysis of variance (ANOVA) was used. Significant ANOVA values (P<0.05) were followed by the Tukey-Kramer post-hoc test for significance to account for unequal group sizes and multiple group testing.

Results

Heterozygous PRCP knockout mice were prepared from KST302 cells obtained from Dr. Williams Skarnes, BayGenomics. DNA from the heterozygous KST302 parent cell line showed significantly more lacZ than ES cells on real time PCR (FIG. 1B). Heterozygous PRCP KO mice were backcrossed 6 generations into a C57BL6 background. Heterozygous PRCP KO mice were then mated and their progeny examined for the presence of lacZ. Animals proposed to be homozygous PRCP KO have significantly increased lacZ expression on real time PCR, whereas littermate controls have lacZ expression at the level of ES cells (FIG. 1B). Mice heterozygous for the PRCP deficiency have lacZ levels comparable to KST302 cells. In mating experiments, mice presumed to be homozygous for PRCP deletion when mated with wild type mice only produced heterozygous animals. Further, mating of heterozygous mice for PRCP deletion with wild type mice only produced heterozygous and wild type mice.

In order to determine the insertional mutation site of the gene-trapped PRCP KO mice, a sense probe from the sequence tag of this PRCP KO cell line from the BayGenomics website and an antisense probe were prepared from the “so-called” TM region of the cloning vector pGT1.8TM. Standard PCR was performed and the determined sequence 3′ to 5′ indicated the TM region was preceded by the splice acceptor (SA) region of the vector that was preceded by the 3′ end of murine PRCP exon 5 (data not shown). These data suggested that the insertion site of the gene trap was between exon and intron 5. Further studies were performed by real time RT PCR using mRNA from KO and wild type mouse kidney, a sense probe from exon 5, an antisense probe from the TM region of the vector, and a detection probe that spans the exon 5-SA region of the vector (Table 4). Kidney RNA of proposed KO mice, as predicted by real time PCR of lacZ, contained exon 5 and the SA-TM regions of the vector on reverse transcribed, real time PCR (FIG. 1C). Alternatively the heterozygous KST302 cells had less RNA of this insertion and kidney RNA from predicted littermate wild type mice or ES cells had none (FIG. 1C).

Further investigations were performed to indicate that distal to the gene trap, no PRCP RNA was produced in mice predicated to be PRCP KO animals by the DNA screening using real time PCR for lacZ (FIG. 1B). Real time RT PCR of kidney RNA from the PRCP KO and wild type mice shown in panels A and B were studied using sense probes from exons 1 or 5 and antisense probes from the vector's TM region or exons 8, 9, and 10 (Table 4, FIG. 1D). The detection probe for these studies was from the contiguous exon 5-vector SA region (Tables 4 and 5). As shown in FIG. 1D, RNA was only seen in the knockout mouse between the exon 1-TM region. No complete RNA from the KO mouse was present using probes from exon 1 through exon 8, exon 1 through exon 10, or exon 5 through exon 9 (FIG. 1D). Further, no exon 5-TM RNA was seen in the littermate wild type mouse. These combined studies indicated that the PRCP KO mouse has its RNA production disrupted after exon 5.

### Example 4

### Identification of PRCP Expression in the Brain

Material and Methods

In situ Hybridization

Mouse PRCP clone EMM1002-16394 from a kidney library prepared by the NCI CGAP program (Cancer Gene Anatomy Program) was purchased from Open Biosciences. Linearized DNA was transcribed using T7 polymerase (antisense cRNA probe) and SP6 polymerase (sense cRNA probe; Riboprobe Combination System SP6/T7, Promega Corporation, Madison, Wis.) and labeled with ³⁵S-UTP (Amersham; 10 mCi/ml). The radiolabelled cRNA probe was then purified by passing the transcription reaction solution over a G50 column (Pharmacia Biotech) and fractions were collected and counted by using a scintillation counter. The purified cRNA probes were heated at 80° C. for 2 minutes with 500 mg/ml yeast tRNA and 50 mM DTT in water before being diluted to an activity of 5.0×10⁷ dpm/ml with hybridization buffer containing 50% formamide, 0.25 M sodium chloride, 1× Denhardt's solution and 10% dextran sulfate. Sections with this hybridization solution (150 ml/slide) were incubated overnight at 50° C. Following hybridization, the slides were washed four times (10 minutes each) in 4×SSC prior to RNase digestion (20 mg/ml for 30 min at 37° C. ), rinsed at room temperature in decreasing concentrations of SSC that contained 1 mM DTT (2×, 1×, 0.5×; 10 minutes each) to a final stringency of 0.1×SSC at 65° C. for 30 minutes (20). After dehydration in increasing alcohols, the sections were exposed to b-max hyperfilm (Amersham) for 5 days before being dipped in Kodak NTB-2 liquid emulsion diluted 1:1 with distilled water. The dipped autoradiograms were developed 21 days later with Kodak D-19 developer, fixed, and the sections counterstained through the emulsion with hematoxylin. Sections were examined under brightfield and darkfield illumination. Several control experiments were carried out to test the specificity of the hybridization method and DII probe. First, sections were incubated as described above with hybridization solution containing the sense-strand probe synthesized by using SP6 polymerase to transcribe the coding strand of the DNA insert. Second, the hybridization was also attempted on sections that had been pretreated with RNase (20 mg/ml for 30 min at 37° C.) to degrade tissue RNA. Third, tissue sections were incubated in radiolabelled probe and then in an excess of unlabeled probe, which competed with the radiolabelled probe, eliminating the increased signal. Moreover, to assess the thermal stability of the hybrid, different series of sections were rinsed in 0.1×SSC at 75° C., 80° C., 85° C., 90° C., 94° C., and 98° C.

X-Gal Staining and Immunocytochemistry

PRCP KO mice were perfused with 4% paraformaldehyde. Brains were sectioned with vibratome (40 μm) and washed with PBS (137 mM NaCl, 2.7 mM KCl, 8 mM Na₂HPO₄, 2.6 mM KH₂PO₄) 4 times. Sections were then rinsed quickly once in cold PBS plus 2 mM MgCl₂ and incubated in the above solution for 10 min at 4° C. Permeabilization was performed by incubation in cold PBS with detergent (0.01% sodium desoxycholate and 0.02% NP40) for 10 min. Sections were then incubated overnight at 37° C. in the staining solution containing 25 mM K₃Fe(CN)₆, 25 mM K₄Fe(CN)₆3H2O, 2 mM MgCl2 in PBS and 1 mg/ml of X-Gal. Sections were then rinsed in PBS and processed for immunocytochemistry. The following antisera were used: mouse b-endorphin, (diluted 1: 5000; Chemicon, Temecula, Calif.) rabbit anti-MCH (diluted 1:12000; Phoenix Pharmaceutics, Belmont, Calif.), goat anti-Hcrt b (diluted 1:3000; Santa Crus Biotechnology, Santa Cruz, Calif.) and mouse anti-thyrosine hydroxylase (TH; diluted 1:5000; Sigma). Antisera were incubated overnight at room temperature. After several washes with PB, sections were incubated in their appropriate secondary antibody (biotinylated goat anti rabbit, horse anti mouse, horse anti goat IgG; 1:250 in PB; Vector Laboratories, Inc., Burlingame, Calif.) for 2 h at room temperature, then rinsed in PB three times 10 min each time, and incubated for 2 h at room temperature with avidin-biotin-peroxidase (ABC, 1:50 in PB; ABC Elite Kit, Vector Laboratories, Inc.). The immunolabeling was visualized with the nickel-diaminobenzidine (DAB) reaction (15 mg DAB, 165 μl 0.3% H₂O₂ in 30 ml PB) for 5-10 min at room temperature resulting in a light brown reaction product.

Results

To clarify the phenotype of PRCP-expressing cells in the hypothalamus, beta galactosidase (β-Gal) expression in hypothalamic slices in PRCP KO mice was analyzed. Expression of β-Gal in the central nervous system was found abundant in various regions, including the cerebral cortex, brains stem, hippocampus and hypothalamus. In the hypothalamus, β-Gal labelled cells were highly expressed in the lateral hypothalamus (LH)-perifornical area and zona incerta (ZI) (FIG. 2A). Labeled cells were also found in the arcuate nucleus, as well as in the dorsalmedial nucleus of the hypothalamus (FIG. 2A). The expression of β-Gal in PRCP KO animals was virtually identical to the distribution pattern of PRCP mRNA in wild type animals as revealed by in situ hybridization of PRCP mRNA (FIG. 2B).

In the arcuate nucleus, the majority of POMC-immunolabeled cells lacked β-Gal expression (FIG. 2) suggesting that if PRCP cleaves α-MSH, it should occur at α-MSH release sites. In hypothalamic regions where PRCP is most abundantly expressed, lateral hypothalamus-perifornical region and dorsomedial nucleus, there are two known orexigenic peptidergic circuits that project to α-MSH -targeted sites: one producing melanin concentrating hormone (MCH) and the other hypocretin/orexin (Hcrt) (Vaughan et al., 1989; Skofitsch et al., 1985; Bittencourt et al., 1992; De Lecea et al., 1998; Sakurai et al., 1998; Trivedi et al., 1998; Horvath et al., 1998; 1999). Double labelling studies revealed that populations of MCH and Hcrt neurons also expressed β-Gal (FIG. 2). Axon terminals of both of these systems, as well as, occasional β-Gal-labelled boutons were found in direct apposition to α-MSH-containing axon terminals in various parts of the hypothalamus, including the paraventricular nucleus (FIG. 2). These observations revealed that hypothalamic PRCP cells are in ideal anatomical position to affect α-MSH action.

### Example 5

### Effect of α-MSH to α-MSH₁₋₁₂ on Food Intake

Material and Methods

ICV Injection of MSH and MSH_{1-12:}

Eighteen male rats (200-250 gr) were used in this study. Animals with a third ventricle cannulation were purchased from Taconic Farms Inc. Rats were single caged and divided in 3 groups: animals injected with saline, animals injected with 2.5 μg of □-MSH and animals injected with 2.5 μg of α-MSH₁₋₁₂. All the rats were fasted overnight. The next morning, the animals were injected and a known amount of food was given and measured after 2 hours from re-feeding (Abbott et al., 2000).

Electrophysiology

Coronal hypothalamic brain slices (˜300 μm) were prepared with a Vibratome from POMC-GFP transgenic mice. Slices were maintained at room temperature in artificial cerebrospinal fluid saturated with 95% O2 and 5% CO2. Slices were equilibrated in the recording chamber for 1 hr at 35° C. in ACSF saturated with 95% O2 and 5% CO2 prior to recordings. ACSF contains (mM): NaCl, 124; KCl, 3; CaCl2, 2; MgCl2, 2; NaH2PO4, 1.23; NaHCO3, 26; glucose, 10; pH 7.4 with NaOH. An upright fluorescence/infrared microscope with long working distance objectives (Olympus BX51WI) and fluorescence filter sets for GFP (Chroma Technology Corp) was used. Fluorescent POMC-GFP neurons were selected and a patch pipette (4-6 mega Ohm resistance) was advanced onto the surface of the neuron by a micromanipulator (Sutter 225). The pipette solution contains (in mM) K gluconate, 128; HEPES, 10; EGTA, 1; KCl, 10; MgCl₂, 1; CaCl₂, 0.3; (Mg)-ATP, 5; (Na)GTP, 0.3, PH.7.4. Whole cell patch clamp experiments were performed with a Multiclamp 700A amplifier (Axon Instruments, Inc). Whole cell current clamp was used to monitor the effect of α-MSH on membrane potential of recorded neurons held at resting membrane potential. All data were sampled at 3-10 kHz and filtered at 1-3 kHz with an Apple Macintosh computer using Axograph 4.9 (Axon Instruments). Eletrophysiological data were analyzed with Axograph 4.9 (Axon Instruments), plotted with Igor Pro software (WaveMetrics, Lake Oswego, Oreg., USA) and presented as the mean±SEM. Statistical analysis was performed by unpaired T test. The level of statistical significance was set at P<0.05.

Results

To examine whether conversion of α-MSH to α-MSH₁₋₁₂ by PRCP could affect food intake, the effect of intracerebroventricular administration of α-MSH and α-MSH₁₋₁₂ on food intake in fasted animals was compared (Abbott et al., 2000). In 12 hour-fasted animals, 2.5 μg of α-MSH induced a 40% reduction in food intake (5.21±0.50 g) compared to the saline-treated animals (8.71±0.56 g; FIG. 3A). On the other hand, 2.5 μg of α-MSH₁₋₁₂ did not significantly affect food intake (8.89±0.90 g) compared to the saline controls (8.71±0.56 gr; FIG. 3A).

The lack of suppression of food intake by α-MSH₁₋₁₂ suggests that the hydrolysis product of α-MSH by PRCP is less active in regulating neuronal functions via melanocortin receptors. To test for that, electrophysiological responses of POMC perikarya to α-MSH and α-MSH₁₋₁₂ was analyzed using patch-clamp in vitro slice recordings from transgenic animals expressing green fluorescence protein (GFP) driven by the mouse POMC promoter (Pinto et al., 2004). This analysis is based on the previously tested phenomenon that activation of MC3 autoreceptors induces hyperpolarization of POMC perikaryal membrane potentials (Cowley et al., 2001). Bath application of α-MSH induced hyperpolarization (66-mV vs. 46-mV) of POMC membrane potential, an effect that was undetectable after application of α-MSH₁₋₁₂ (FIG. 3B). This electrophysiological analyzes confirmed that the hydrolysis product of α-MSH by PRCP is ineffective in triggering adequate neuronal responses.

### Example 6

### Inhibition of PRCP

Material and Methods

ICV Injection of B-PP

Twelve male rats (200-250 gr) were used in this study. Animals with a third ventricle cannulation were purchased from Taconic Farms Inc. Rats were single caged and divided in 2 groups: animals injected with 0.9 μg of B-PP and animals injected with the same volume of vehicle. A known amount of food was given to the animals and weighted every 24 hours.

IP Injection of B-PP

For this study 10 ob/ob mice were used. Mice were injected at the beginning of the dark phase intraperitoneally with 2 doses of 400 pg of B-PP every 24 hours and food intake was measured before each injection and every 24 hours after the last administration. Control mice were injected at the same times and with same volume of vehicle.

IP Injection of Z-PP

For this study 10 ob/ob mice were used. Mice were injected at the beginning of the dark phase intraperitoneally with 2 doses of 5 mg Z-PP every 12 hours and food intake was measured every 12 hours after the last injection. Control mice were injected at the same times and with the same volume of vehicle. Note that for Z-PP doses lower than 5 mg were ineffective in reducing food intake. Moreover, the lasting effect was also reduced (after 24 hours from the last injection there was no difference in food intake compared to vehicle controls).

IP Administration of α-MSH and MTII to PRCP KO and Wild Type Mice

Wild type and PRCP KO mice were used in this study. Animals were single caged and injected with saline at the beginning of the dark phase for 3 days. For each mouse, food intake was measured at 4, 12 and 24 hours after the injection. After 3 days, animals were divided in 6 groups: 1) wild type mice receiving 200 nmol MTII in saline; 2) wild type mice receiving 200 nmol α-MSH in saline; 3) wild type mice receiving saline; 4) PRCP KO mice receiving 200 nmol of MTII in saline; 5) PRCP KO mice receiving 200 nmol α-MSH; 6) PRCP KO mice receiving saline.

Results

If PRCP-regulated hydrolysis of α-MSH has a physiological role in regulation of food intake, then inhibition of PRCP enzymatic activity should interfere with feeding behaviour. To test this, the effects of two previously characterized inhibitors of PRCP, t-Butyl Carbamate (BOC)-prolyl prolinal (B-PP) and N-benzyloxycarbonyl-prolyl-prolinal (Z-PP) (Wilk et al., 1983; Friedman et al., 1984) was analyzed on food intake. Intracerebroventricular administration of B-PP (0.9 mg) to rats induced suppression of overnight food intake (36.7% versus 94.91% of controls after 24 hours from the administration), which was followed by recovery to controls levels without detectable rebound feeding (89.24% versus 97.67% of controls after 48 hours from the administration (FIG. 3C). After administration of B-PP systemically (400 μg) to obese, leptin deficient (ob/ob) mice it was found that B-PP suppressed food intake and body weight gain of these genetically obese animals (FIG. 3D). The other inhibitor of PRCP, Z-PP, was also tested in ob/ob animals (5 mg ip) and was found to suppress food intake, but with lower efficacy than that of B-PP (FIG. 3E). Nevertheless, these observations showed that targeting PRCP activity with central or peripheral administration of protease inhibitors can reduce food intake and body weight gain.

Despite of α-MSH's well described anorectic function and the recognition of MC4R as critical mediator of melanocortins' food reducing effects (Fan et al., 1997), peripheral administration of α-MSH has not been reported to reduce food intake. In contrast, a synthetic analog of melanocortins, MTII (Ac-Nle⁴-c[Asp⁵, D-Phe⁷, Lys¹⁰] α-MSH-(4-10)-NH₂) is readily able to reduce food intake even after peripheral administration (Fan et al., 1997). The diminished efficacy of peripheral α-MSH on food intake together with the recent discovery of α-MSH variants that are more stable Guo et al., 1997), suggest that extracellular degradation of α-MSH is an important element in determining its anorexigenic efficacy. To test whether PRCP masks the anorectic effects of peripheral α-MSH administration, the effect of peripherally administered α-MSH on food intake regulation in wild type and PRCP KO mice were tested and the values were compared to those obtained by peripheral MTII administration. Intraperitoneal injection of α-MSH (200 nmol) into wild type mice had no effect on feeding (FIG. 4), while MTII (200 nmol) administration resulted in significant decline in food intake (35.5% food intake compared to 100% controls; FIG. 4). However, ip. injection of α-MSH (200 nmol) into PRCP-ablated mice strikingly had an equivalent effect of food intake reduction as MTII (34.3% food intake compared 100% controls; FIG. 4). Because PRCP is expressed in various peripheral tissues as well as in the brain, it is reasonable to suggest that PRCP is responsible for inactivation of peripherally injected α-MSH regarding food intake regulation.

In summary, the B6.C-D7MIt353 congenic with a maximum of 14.7 megabases of BALB/cJ donor DNA on a C57BL/6J background (see Table 3) includes at least one statistically significant functional obesity candidate gene-Prcp. Survey of all other genes in the donor region using a microarray with complete coverage revealed no other genes with statistically significant differential expression. The discovery that PRCP is a positional candidate obesity gene, despite its being located in a haplotype shared by B6 and the BALB/c donor strain, motivated mechanistic studies which have now shown that it is a member of a validated human obesity pathway. The data disclosed herein demonstrate that pharmacological inhibition of PRCP could be used to successfully combat obesity and related disorders.

The foregoing detailed description has been given for clearness of understanding only and no unnecessary limitations should be understood therefrom as modifications will be obvious to those skilled in the art.

While the invention has been described in connection with specific embodiments thereof, it will be understood that it is capable of further modifications and this application is intended to cover any variations, uses, or adaptations of the invention following, in general, the principles of the invention and including such departures from the present disclosure as come within known or customary practice within the art to which the invention pertains and as may be applied to the essential features hereinbefore set forth and as follows in the scope of the appended claims.

### REFERENCE

The disclosures of each and every patent, patent application, and publication cited herein including but limited to the references listed immediately below are hereby incorporated herein by reference in their entirety.

### REFERENCES

1. Vaisse, C. et al. Melanocortin-4 receptor mutations are a frequent and heterogeneous cause of morbid obesity. J. Clin Invest 106, 253-62 (2000).
2. Krude, H. et al. Severe early-onset obesity, adrenal insufficiency and red hair pigmentation caused by POMC mutations in humans. Nat Genet 19, 155-7 (1998).
3. Naggert, J. K. et al. Hyperproinsulinaemia in obese fat/fat mice associated with a carboxypeptidase E mutation which reduces enzyme activity. Nat Genet 10, 135-42 (1995).
4. Duhl, D. M., Vrieling, H., Miller, K. A., Wolff, G. L. & Barsh, G. S. Neomorphic agouti mutations in obese yellow mice. Nat Genet 8, 59-65 (1994).
5. Jackson, R. S. et al. Obesity and impaired prohormone processing associated with mutations in the human prohormone convertase 1 gene. Nat Genet 16, 303-6 (1997).
6. Huszar D, Lynch C A, Fairchild-Huntress V, Dunmore J H, Fang Q, Berkemeier L R, Gu W, Kesterson R A, Boston B A, Cone R D, Smith F J, Campfield L A, Burn P, Lee F 1997 Targeted disruption of the melanocortin-4 receptor results in obesity in mice. Cell 88:131-141.
7. Diament, A. L. & Warden, C. H. Multiple linked mouse chromosome 7 loci influence body fat mass. Int J Obes Relat Metab Disord (2003).
8. McCarthy, J. J., Meyer, J., Moliterno, D. J., Newby, L. K., Rogers, W. J., Topol, E. J. GenQuest multicenter study. Evidence for substrantial effect modification by gender in a large scale genetic association study of the metabolic syndrome among coronary heart disease patients. Hum Genet. 114, 87-98 (2003)
9. Shariat-Madar, Z., Mahdi, F., Schmaier, A. H. Identification and characterization of prolylcarboxypeptidase as an endothelial cell prekallikrein activator. J. Biol. Chem. 277, 17962-17969 (2002)
10. Shariat-Madar, Z., Mahdi, F., Schmaier, A. H. Recombinant prolylcarboxypeptidase activates plasma prekallikrein. Blood. 103, 4554-4561 (2004).
11. Odya, C. E., Marinkovic, D. V., Hammon, K. J., Stewart, T. A., Erdos, E. G. Purification and properties of prolylcarboxypeptidase (Angiotensinase C) from human kidney. J. Biol. Chem. 253, 5927-5931 (1978).
12. Jacobowitz D M, O'Donohue T L 1978 α-Melanocyte stimulating hormone: immunohistochemical identification and mapping in neurons of the rat brain. Proc Natl Acad Sci USA 75:6300-6304.
13. Mountjoy K G, Mortrud M T, Low M J, Simerly R B, Cone R D 1994 Localization of the melanocortin-4 receptor (MC4-R) in neuroendocrine and autonomic control circuits in the brain. Mol Endocrinol 8:1298-1308.
14. Tatro J B 1990 Melanotropin receptors in the brain are differentially distributed and recognize both corticotropin and -melanocyte stimulating hormone. Brain Res 536:124-132.
15. Vaughan J M, Fischer W H, Hoeger C, Rivier J, Vale W 1989 Characterization of melanin-concentrating hormone from rat hypothalamus. Endocrinology 125:1660-1665.
16. Skofitsch G, Jacobowitz D M, Zamir N 1985 Immunohistochemical localization of a melanin concentrating hormone-like peptide in the rat brain. Brain Res Bull 15:635-649.
17. Bittencourt J C, Presse F, Arias C, Peto C, Vaughan J, Nahon J L, Vale W, Sawchenko PE 1992 The melanin-concentrating hormone system of the rat brain: an immuno- and hybridization histochemical characterization. J Comp Neurol 319:218-245.
28. De Lecea L, Kilduff T S, Peyron C, Gao X, Foye P E, Danielson P E, Fukuhara C, Battenberg E L, Gautvik V T, Bartlett II F S, Frankel W N, van den Pol A N, Bloom FE, Gautvik K M, Sutcliff J G 1998 The hypocritins: hypothalamic-specific peptide with neuroexicitatory activity. Proc Natl Acad Sci USA 95:322-327.
19. Sakurai T, Amemiya A, Ishii M, Matsuzaki I, Chemelli R M, Tanaka H, Williams S C, Richardson J A, Kozlowski G P, Wilson S, Arch J R, Buckingham R E, Haynes A C, Carr S A, Annan R S, McNulty D E, Liu W S, Terrett J A, Elshourbagy N A, Bergsma D J, Yanagisawa M 1998 Orexins and orexin receptors: a family of hypothalamic neuropeptides and G protein-coupled receptors that regulate feeding behavior. Cell 92:573-585.
20. Trivedi P, Yu H, MacNeil D, Van der Ploeg L H T, Guan X-M 1998 Distribution of orexin receptor mRNA in the rat brain. FEBS Lett 438:71-75.
21. Horvath T L, Diano S, van den Pol AN Hypocretin (orexin)-containing neurons make synaptic contact with arcuate nucleus NPY and POMC-producing cells that express leptin receptors in rodents and primates: novel hypothalamic circuit involved in energy homeostasis. Program of the 28th Annual Meeting of the Society for Neuroscience, Los Angeles, Calif., 1998, vol 24:12 (Abstract).
22. Horvath T L, Diano S, van den Pol A N 1999 Synaptic interaction between hypocretin (orexin) and neuropeptide Y cells in the rodent and primate hypothalamus: a novel circuit implicated in the metabolic and endocrine regulation. J Neurosci 19:1072-1087.
23. Abbott C R, Rossi M, Kim M, AlAhmed S H, Taylor G M, Ghatei M A, Smith D M, Bloom S R. 2000 Investigation of the melanocyte stimulating hormones on food intake. Lack Of evidence to support a role for the melanocortin-3-receptor. Brain Res. 869:203-10.
24 Pinto S, Roseberry A G, Liu H, Diano S, Shanabrough M, Cai X, Friedman J M, Horvath T L 2004 Rapid rewiring of arcuate nucleus feeding circuits by leptin. Science 304:110-115.
25 Cowley M A, Smart J L, Rubistein M, Cerdan M G, Diano S, Horvath T L, Cone R D, Low M J (2001) Leptin activates anorexigenic POMC neurons through a neuronal network in the arcuate nucleus. Nature 411:480-484.
26. Wilk S, Orlowski M 1983 Inhibition of rabbit prolyl endopeptidase by N-benzyloxycarbonyl-Prolyl-Prolinal, a transition state aldehydes inhibitor. J Neurochemistry 41:69-75.
27. Friedman T C, Orlowski M, Wilk S 1984 Prolyl endopeptidase: inhibition in vivo by N-benzyloxycarbonyl-Prolyl-Prolinal. J Neurochemistry 42:237-241.
28. Fan W, Boston B A, Kesterson R A, Hruby V J, Cone R D 1997 Role of melanocortinergic neurons in feeding and the agouti obesity syndrome. Nature 385:119-20.
29. Guo L, Munzberg H, Stuart R C, Nillni E A, Bjorbaek C. 2004 N-acetylation of hypothalamic alpha-melanocyte-stimulating hormone and regulation by leptin. Proc Natl Acad Sci USA. 101:11797-802.
30. Skarnes W C, Moss J E, Hurtley S M, Beddington RSP. 1995 Capturing genes encoding membrane and secreted proteins important for mouse development. Proc. Natl Acad. Sci 92:6592-6596.
31. Schmittgen T D, Zakrajsek B A, Mills A G, Gorn V, Singer M J. 2000 Quantitative reverse transcription-polymerase chain reaction to study mRNA decay: comparison of endpoint and real-time methods. Anal Biochem. 285:194-204.

TABLE 1
Congenic Region Genes with LPE pval <0.01

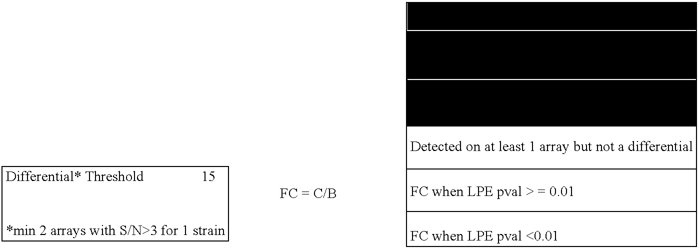

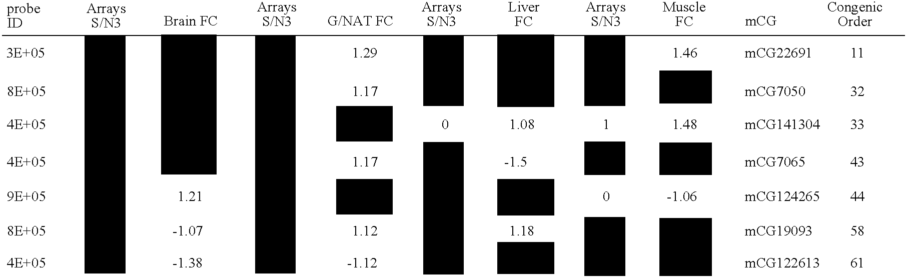

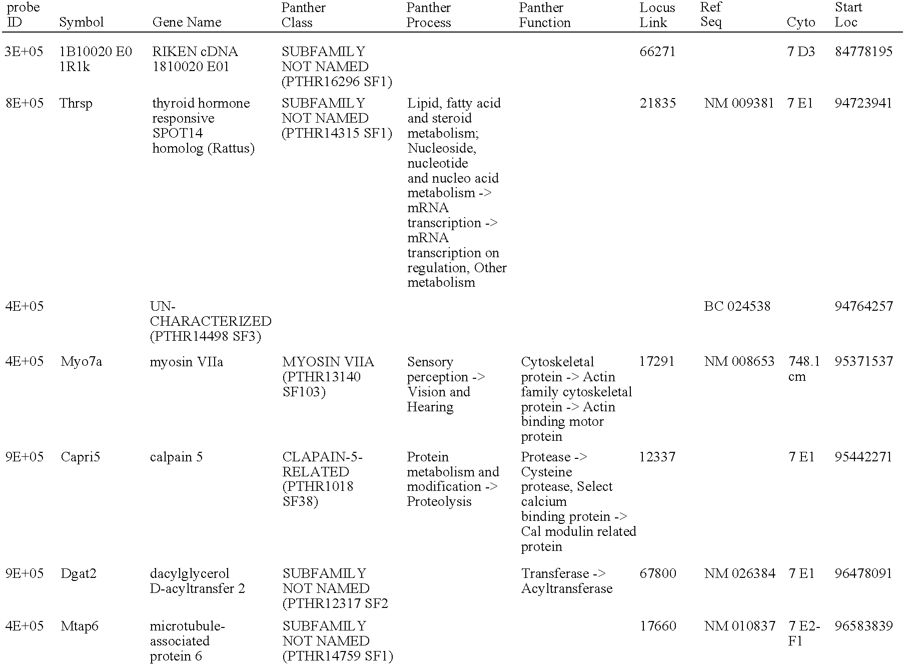

TABLE 2

Prcp sequencing primers and quantitative PCR
primers

| | | |
|---|---|---|
| Prcp | 1upstreamproxf: | SEQ ID |
| Sequencing | 5′-CAAAGGACACAGACAAGCCC-3′ | NO: 13 |
| | 1upstreamproxr: | SEQ ID |
| | 5′-CCACACTCAACCAGAACAGC-3′ | NO: 14 |
| | 2upstreamproxf: | SEQ ID |
| | 5′-GTCCAACTCCGTCTGGAAGG-3′ | NO: 15 |
| | 2upstreamproxr: | SEQ ID |
| | 5′-AAGACAGGAGCAGGAGAGCC-3′ | NO: 16 |
| | 5′UTRproxf: | SEQ ID |
| | 5′-TGCCTGGTTTTAGGTTTCCC-3′ | NO: 17 |
| | 5′UTRproxr: | SEQ ID |
| | 5′-AAGACAGGAGCAGGAGAGCC-3′ | NO: 16 |
| | 1proxf: | SEQ ID |
| | 5′-CTTTAGTCGCCCACACTGTG-3′ | NO: 18 |
| | 1proxr: | SEQ ID |
| | 5′-CAGGTTCACCCAGGTCTCAG-3′ | NO: 19 |
| | 2proxf: | SEQ ID |
| | 5′-CAGCCTGGTTCAGGATGAAG-3′ | NO: 20 |
| | 2proxr: | SEQ ID |
| | 5′-CACAGAGGAAGGATCGAAGG-3′ | NO: 21 |
| | 3proxf: | SEQ ID |
| | 5′-CTATCAACATCCATGACGGGGC-3′ | NO: 22 |
| | 3proxr: | SEQ ID |
| | 5′-TTGGCAGAATGTAGCCAGAC-3′ | NO: 23 |
| | 3′UTRproxf: | SEQ ID |
| | 5′-GCCTTGTCCTGCTCTCTTTG-3′ | NO: 24 |
| | 3′UTRproxr: | SEQ ID |
| | 5′-AAGTCCCCACACACCTGATG-3′ | NO: 25 |
| Sybr Primers | prcpabif: | SEQ ID |
| for Prcp | 5′-GAACTACCCTTACGCATGCAACT-3′ | NO: 26 |
| | prcpabir: | SEQ ID |
| | 5′-AATATTGGCACACCTCCTTGATG-3′ | NO: 27 |
| Sybr primers | AbrpF: | SEQ ID |
| for Abrp | 5′-GGACCCGAGAAGAGCTCCTCCTT-3′ | NO: 28 |
| | AbrpR: | SEQ ID |
| | 5′-TCAATGGTGCCTCTGGAGATT-3′ | NO: 29 |

TABLE 3

| Panther Function | LocusLink | RefSeq | Cyto | Start Loc | Congenic Order |
|---|---|---|---|---|---|
| | | AK014876 | | 84183929 | 1 |
| | 67669 | NM_026304 | 7 D3 | 84236719 | 2 |
| Transcription factor -> Other | 13626 | NM_021876 | 7 47.0 cM | 3 | 3 |
| transcription factor | | | | | |
| | | AK046045 | | 84351921 | 4 |
| Transcription factor -> Basal | | | | 84432318 | 5 |
| transcription factor | | | | | |
| | | AK019082 | | 84449997 | 6 |
| Membrane traffic protein -> | 233489 | NM_146194 | 7 47.2 cM | 84455387 | 7 |
| Vesicle coat protein | | | | | |
| Membrane traffic protein -> | 83671 | NM 031394 | 7 D3 | 84629289 | 8 |
| Membrane traffic regulatory | | | | | |
| protein | | | | | |
| | | AK006105 | | 84753586 | 9 |
| | 233490 | | 7 D3 | 84770337 | 10 |
| | 66271 | | | 84778195 | 11 |
| | | BC049680 | | 84795733 | 12 |
| | | AK046356 | | 84804627 | 13 |
| Cytoskeletal protein -> Actin | 23859 | NM 011807 | 7 D3 | 85763169 | 14 |
| family cytoskeletal protein Cell | | | | | |
| junction protein -> Other cell | | | | | |
| junction protein | | | | | |
| | | | | 86855206 | 15 |
| | 73845 | NM 028665 | 7 D3 | 86881740 | 16 |
| Nucleic acid binding -> | | NM 029078 | | 86934924 | 17 |
| Nuclease; Nucleic acid | | | | | |
| binding -> MrnA processing | | | | | |
| factor | | | | | |
| Select regulatory molecule -> | 75985 | NM 029494 | 7 D3 | 87932620 | 18 |
| G-protein -> Small GTPase | | | | | |
| | | NM 028729 | | 87141999 | 19 |
| Protease -> Serine protease | 72461 | NM 028243 | 7 D3 | 87163575 | 20 |
| | | | | 87219883 | 21 |
| | 58238 | NM 021427 | 7 D3 | 87289017 | 22 |
| | | | | 88313547 | 23 |
| Protease -> Other proteases | | AK034106 | | 88655262 | 24 |
| Viral protein-> Other viral | | | | | |
| protein | | | | | |
| | | | | 88655243 | 25 |
| Nucleic acid binding -> Other | | | | 89737851 | 26 |
| RNA-binding protein | | | | | |
| | 66365 | NM 025515 | 7 | 91033224 | 27 |
| | | AK077305 | | 91010506 | 28 |
| Nucleic acid binding -> | 15012 | NM 008251 | 16 69.8 cM | 94669420 | 29 |
| Chromatin/chromatin-binding | | | | | |
| protein | | | | | |
| Transferase | 381903 | NM_199035 | 7 E1 | 94676128 | 30 |
| Oxidoreductase | 68197 | NM_024220 | 7 E1 | 94711155 | 31 |
| | 21835 | NM_009381 | 7 E1 | 94723941 | 32 |
| | | BC024588 | | 94764257 | 33 |
| | 101861 | NM_027256 | 7 E1 | 94793837 | 34 |
| | 66273 | NM_183251 | 7 E1 | 94865537 | 35 |
| | | BC054454 | | 94898008 | 36 |
| | | AK005667 | | 94916464 | 37 |
| | | AK041721 | | 94969805 | 38 |
| Transporter -> Other | 12729 | NM_023671 | 7 50.0 cM | 95016473 | 39 |
| transporter | | | | | |
| | 66333 | NM_175105 | 7 E1 | 95046310 | 40 |
| Kinase -> Protein kinase -> | 18479 | AK078711 | 7 46.5 cM | 95110313 | 41 |
| Non-receptor serine/threonine | | | | | |
| protein kinase | | | | | |
| | 233537 | NM_177696 | 7 E1 | 95241025 | 42 |
| Cytoskeletal protein -> Actin | 17921 | NM_008663 | 7 48.1 Cm | 95371537 | 43 |
| family cytoskeletal protein -> | | | | | |
| Actin binding motor protein | | | | | |
| Protease -> Cysteine | 12337 | | 7E1 | 95442271 | 44 |
| proteaseSelect calcium | | | | | |
| binding protein -> Calmodulin | | | | | |
| related protein | | | | | |
| Signaling molecule -> Other | 18378 | NM_011010 | 7 48.0 cM | 95465226 | 45 |
| signaling molecule | | | | | |
| | 330599 | NM_177896 | 7E1 | 95498522 | 46 |
| Transferase -> | | BC039789 | | 95514484 | 47 |
| Glycosyltransferase | | | | | |
| | | AK053951 | | 95527630 | 48 |
| Receptor | | AK035461 | | 95671996 | 49 |
| Oxidoreductase | | BC059730 | | #REF! | 50 |
| Lyase -> Cyclase -> | | | | 95766435 | 51 |
| Guanylate cyclase | | | | | |
| Defense/immunity protein -> | | | | 95817397 | 52 |
| Antibacterial response protein | | | | | |
| | 233545 | NM_172280 | 7E1 | 95908991 | 53 |
| | | AK052909 | | 96006624 | 54 |
| | 72981 | | 7F1 | 96024131 | 55 |
| Signaling molecule -> Other | 22411 | NM_009519 | 7 48.0 cM | 96156716 | 56 |
| signaling molecule | | | | | |
| | 78610 | AK020590 | 7E1 | 96208474 | 57 |
| Transferase -> | 67800 | NM_026384 | 7E1 | 96478091 | 58 |
| Acyltransferase | | | | | |
| Transferase -> | 233549 | NM_177448 | 7E1 | 96544118 | 59 |
| Acyltransferase | | | | | |
| | | BC050000 | | 96586291 | 60 |
| | 17760 | NM_010837 | 7 E2-F1 | 96593839 | 61 |
| ChaperoneSelect regulatory | 12406 | NM_009825 | 7E1 | | 62 |
| molecule -> Protease inhibitor | | | | | |
| -> Serine protease inhibitor | | | | | |
| | 233552 | | | 96706470 | 63 |
| | AK013012 | | | 96790689 | 64 |
| Nucleic acid binding -> | 27050 | | 7 49.6 cM | 96802892 | 65 |
| Ribosomal protein | | | | | |
| Miscellaneous function -> | 109689 | NM_178220 | 7 50.0 cM | 96859831 | 66 |
| Other miscellaneous function | | | | | |
| protein | | | | | |
| Cell adhesion molecule | | AK032609 | | 96950950 | 67 |
| Transporter -> Other | 101488 | NM_175316 | 7E1 | 96983703 | 68 |
| transporter | | | | | |
| Receptor -> G-protein coupled | 259066 | NM_147063 | | 97058092 | 69 |
| receptor | | | | | |
| Receptor -> G-protein coupled | 258353 | 258353 | | 97090113 | 70 |
| receptor | | | | | |
| Hydrolase -> Other hydrolase | 50877 | NM_016720 | | 97136643 | 71 |
| Protease | 66624 | | 7 E1 | 97163797 | 72 |
| | | AK040058 | | 97197602 | 73 |
| | | | | 97201983 | 74 |
| | | | | 97252039 | 75 |
| Ligase -> Other ligase | 67164 | | 7 E1 | 97493860 | 76 |
| Ion channel -> Voltage-gated | 57442 | | 7 E1 | 97513242 | 77 |
| ion channel -> Voltage-gated | | | | | |
| potassium channel | | | | | |
| Isomerase -> Mutase | 70974 | NM_027629 | 7 E1 | 97562568 | 78 |
| Oxidoreductase -> | 320452 | NM_177161 | 7 E1 | 97621543 | 79 |
| Hydroxylase | | | | | |
| Hydrolase -> Esterase | 72590 | NM_028292 | 7 E1 | 97664564 | 80 |
| | | BC046540 | | 97709402 | 81 |
| | | | | 97763713 | 82 |
| Chaperone -> Other | 69387 | NM_153527 | 7 E1 | 97839315 | 83 |
| chaperones | | | | | |
| | 68185 | NM_183270 | 7 E1 | 97875299 | 84 |
| | 52443 | NM_198831 | 4 42.5 cM | 97887139 | 85 |
| Select regulatory molecule -> | 19346 | NM_024287 | 7 E1 | 97946564 | 86 |
| G-protein -> Small GTPase | | | | | |
| Membrane traffic protein -> | 27276 | NM_013746 | 7 E1 | 97983081 | 87 |
| Other membrane traffic | | | | | |
| protein | | | | | |
| Receptor -> Cytokine receptor | 320100 | NM_177073 | 7 E1 | 98196483 | 88 |
| -> Tumor necrosis factor | | | | | |
| receptor | | | | | |
| | | BC028887 | | 98220370 | 89 |
| Receptor -> G-protein coupled | 233571 | NM_18316 | 7 E1 | 98288704 | 90 |
| receptor | | | | | |
| | 207278 | NM_199012 | 7 E1 | 98462084 | 91 |
| Nucleic acid binding -> | | | | 98507948 | 92 |
| Ribosomal protein | | | | | |
| | | BC060054 | | 98645698 | 93 |
| Transfer/carrier protein -> | 56018 | | 7 E1 | 98675709 | 94 |
| Other transfer/carrier protein | | | | | |
| | 69710 | NM_027180 | | 98706605 | 95 |
| Hydrolase -> | | BC006845 | | 98780598 | 96 |
| Phosphodiesterase | | | | | |
| Transferase -> | 11871 | NM_007490 | 7 49.0 cM | 98916271 | 97 |
| Glycosyltransferase | | | | | |

TABLE 4

Primers for the Characterization of the PRCP KO Mice

| | | Position | | Position | | Position |
|---|---|---|---|---|---|---|
| | | On | | On | | On |
| | | Vector | | Vector | | Vector |
| Target | Sense Primer | or cDNA | Antisense primer | or cDNA | Probe # | or cDNA |
| Geno- | | | | | | |
| typing | | | | | | |
| B-lac Z | 5′-ATGGCGATTACCGTTGATGT-3′ | 5146- | 5′- | 5247- | 5′-56-FAM/ | 5167-5185 |
| | (SEQ ID NO: 2) | 5165 | CCAGTTTACCCGCTCTGCTA- | 5228 | GAAGTGGCGAGCGATACAC/ | |
| | | | 3′ | | 3BHQ1/-3′ | |
| | | | (SEQ ID NO: 3) | | (SEQ ID NO: 4) | |
| Inser- | | | | | | |
| tional | | | | | | |
| mutation | | | | | | |
| site | | | | | | |
| E5-TM | 5′-GGCATGCTTGCAGCCTGGTT-3′ | 606-626 | 5′- | 1952- | 5′-56-FAM/ | 621-630: |
| | (SEQ ID NO: 5) | | GTGATCCAGGACTGGGAAGA- | 1932 | TTGTAGTTGGGTCCCAGGTC/ | 1715-1725 |
| | | | 3′ | | 3BHQ1/-3′ | |
| | | | (SEQ ID NO: 6) | | (SEQ ID NO: 7) | |
| PRCP | | | | | | |
| mRNA | | | | | | |
| E1-TM | 5′-TGCTGGTGGCTCTCTTTCT-3′ | 16-34 | 5′- | 1952- | 5′-56-FAM/ | 621-630: |
| | (SEQ ID NO: 8) | | GTGATCCAGGACTGGGAAGA- | 1932 | TTGTAGTTGGGTCCCAGGTC/ | 1715-1725 |
| | | | 3′ | | 3BHQ1/-3′ | |
| | | | (SEQ ID NO: 6) | | (SEQ ID NO: 7) | |
| E1-E8 | 3′-TGCTGGTGGCTCTCTTTCT-3′ | 16-34 | 5′- | 1150- | 5′-56-FAM/ | 621-630: |
| | (SEQ ID NO: 8) | | AAAGGGCATGACCATTTCTG- | 1131 | TTGTAGTTGGGTCCCAGGTC/ | 1715-1725 |
| | | | 3′ | | 3BHQ1/-3′ | |
| | | | (SEQ ID NO: 9) | | (SEQ ID NO: 7) | |
| E1-E10 | 5′-TGCTGGTGGCTCTCTTTCT-3′ | 16-34 | 5′- | 1333- | 5′-56-FAM/ | 621-630: |
| | (SEQ ID NO: 8) | | CCACGGGTCTAACTCACCAT- | 1314 | TTGTAGTTGGGTCCCAGGTC/ | 1715-1725 |
| | | | 3′ | | 3BHQ1/-3′ | |
| | | | (SEQ ID NO: 10) | | (SEQ ID NO: 7) | |
| E5-E9 | 5′- | 496-520 | 5′- | 1195- | 5′-56-FAM/ | 621-630: |
| | TTTTGCAGAGTTAATCAGACACTTG- | | GTCCCACAGAAAGGGTTCAA- | 1176 | TTGTAGTTGGGTCCCAGGTC/ | 1715-1725 |
| | 3′ | | 3′ | | 3BHQ1/-3′ | |
| | (SEQ ID NO: 11) | | (SEQ ID NO: 12) | | (SEQ ID NO: 7) | |

Genotyping by real time PCR was performed with tail DNA with the indicated primers. The characterization of the insertional mutation site was performed with mRNA from a wild type and KO mouse.
“E” stands for exon region on PRCP; “TM” stands for the TM region on trapping vector pGT1.8TM.
* indicates two numbering locations: the smaller numbers are the location of the 3′ end of exon 5 from PRCP cDNA; the larger numbers indicate the numbering of the SA region on the vector pGT1.8TM. These regions are contiguous in the PRCP gene of gene-trapped mice.

TABLE 5

| | | | | | | | Delta Delta | | | | | |
|---|---|---|---|---|---|---|---|---|---|---|---|---|
| | | | | | | | (Prcp - Control) | | | | | |
| | Ct | | | Genotype | | | (C − B) - | | (C − H) - | | (H − B) - | |
| | values | | | effects | | | Average | fold | Average | fold | Average | fold |
| | B | C | H | C − B | C − H | H − B | control | B/C | control | H/C | control | B/H |
| Prcp by Sybr green | 27.81 | 28.82 | 28.66 | 1.01 | 0.16 | 0.85 | 0.97 | 1.95 | 0.66 | 1.58 | 0.31 | 1.24 |
| Exon 2 | 31.56 | 32.14 | 31.50 | 0.58 | 0.64 | −0.06 | 1.05 | 2.07 | 0.65 | 1.57 | 0.40 | 1.32 |
| Exon 3 | 30.15 | 30.97 | 30.84 | 0.82 | 0.13 | 0.69 | 0.78 | 1.71 | 0.63 | 1.55 | 0.15 | 1.11 |
| Exon 5 | 29.49 | 30.26 | 30.35 | 0.77 | −0.09 | 0.86 | 0.73 | 1.65 | 0.41 | 1.33 | 0.32 | 1.24 |
| 36B4 by Sybr | 20.39 | 20.30 | 20.93 | −0.09 | −0.63 | 0.54 | | | | | | |
| Beta-Glucuronidase | 27.97 | 28.12 | 28.53 | 0.15 | −0.41 | 0.56 | | | | | | |
| Beta-2-microglobulin | 22.95 | 22.81 | 23.48 | −0.14 | −0.67 | 0.53 | | | | | | |
| TBP | 26.23 | 26.35 | 26.95 | 0.12 | −0.60 | 0.72 | | | | | | |
| Glyceraldehyde | 20.73 | 20.91 | 21.10 | 0.18 | −0.19 | 0.37 | | | | | | |
| phosphate | | | | | | | | | | | | |
| dehydrogenase | | | | | | | | | | | | |
| Beta-2-microglobulin | 29.12 | 28.65 | 28.66 | −0.47 | −0.01 | −0.46 | | | | | | |
| (assay for Exon 2) | | | | | | | | | | | | |

Exon 2 = ABI TaqMan assay number 1324043
Exon 3 = ABI assay number 804504
Exon 5 = ABI assay number 804505

1. A method of preventing or treating obesity and obesity-related disorders in an animal in need thereof, said method comprising administering to the animal a prolylcarboxypeptidase (PRCP) inhibiting amount of a PRCP inhibitor.

2. The method of claim 1, wherein said PRCP inhibitor is selected from the group consisting of a ribozyme, a siRNA, an antisense nucleic acid, an aptamer, an antibody, a peptide and a chemical compound.

3. The method of claim 2, wherein said chemical compound is selected from the group consisting of t-butyl carbamate (BOC)-prolyl prolinal (B-PP), N-benzyloxycarbonyl-prolyl-prolinal (Z-PP), diisopropyl fluorophosphate, PMSF, antipain, leupeptin, corn trypsin, and mercuric chloride.

4. The method of claim 3, wherein said chemical compound is selected from the group consisting of B-PP and Z-PP.

5. The method of claim 2, wherein said peptide is selected from the group consisting of α-melanocyte stimulating hormone (α-MSH) and angiotensinogin II (AgII).

6. The method of claim 1, wherein said PRCP inhibitor is a PRCP substrate.

7. The method of claim 1, wherein the animal suffers from or is predisposed to obesity.

8. The method of claim 1, wherein the animal is obese and suffers from or is predisposed to one or more obesity-related disorders selected from the group consisting of hypertension, dyslipidemia, diabetes, stroke, gallbladder disease, cardiovascular disease, osteoarthritis, rheumatoid arthritis, hypercholesterolemia, stable angina, unstable angina, sleep apnea, respiratory problems, cancer, stroke hyerinsulinemia, Syndrome X, hypercholesterolemia, hyperlipoproteinernia, hypertriglyceridemia, atherosclerosis, diabetic renal disease.

9. The method of claim 8, wherein the disorder is diabetes.

10. A method for inducing weight loss, decreasing body fat and/or decreasing food intake in an animal in need thereof, said method comprising administering to the animal a PRCP inhibiting amount of a PRCP inhibitor, thereby inducing weight loss, decreasing body fat, and/or decreasing food intake in said animal.

11. (canceled)

12. The method of claim 10, wherein said animal is selected from the group consisting of a bird, a rodent, and a mammal, and further wherein said mammal is selected from the group consisting of a cow, a pig, a sheep, a buffalo, a beefalo, a bison, a deer, a goat, and a human.

13. (canceled)

14. A method for affecting appetite. affecting physical activity, and/or affecting metabolic rate in an animal in need thereof, said method comprising administering a PRCP inhibiting amount of a PRCP inhibitor to the animal, thereby affecting appetite, affecting physical activity and/or affecting metabolic rate in said animal.

15-16. (canceled)

17. The method of claim 1, wherein the PRCP inhibitor is administrated peripherally.

18. The method of claim 1, wherein the PRCP inhibitor is administrated centrally.

19-26. (canceled)


SEQUENCE LISTING (31 sequences)

SEQ ID NO: 1 - protein, 13 aa, Homo sapiens
SYSMEHFRWGKPV

SEQ ID NO: 2 - DNA, 20 bp, Artificial Sequence
  Primer
atggcgattaccgttgatgt

SEQ ID NO: 3 - DNA, 20 bp, Artificial Sequence
  Primer
ccagtttacccgctctgcta

SEQ ID NO: 4 - DNA, 19 bp, Artificial Sequence
  Probe
gaagtggcgagcgatacac

SEQ ID NO: 5 - DNA, 20 bp, Artificial Sequence
  Primer
ggcatgcttgcagcctggtt

SEQ ID NO: 6 - DNA, 20 bp, Artificial Sequence
  Primer
gtgatccaggactgggaaga

SEQ ID NO: 7 - DNA, 20 bp, Artificial Sequence
  Probe
ttgtagttgggtcccaggtc

SEQ ID NO: 8 - DNA, 19 bp, Artificial Sequence
  Primer
tgctggtggctctctttct

SEQ ID NO: 9 - DNA, 20 bp, Artificial Sequence
  Antisense primer
aaagggcatgaccatttctg

SEQ ID NO: 10 - DNA, 20 bp, Artificial Sequence
  Primer
ccacgggtctaactcaccat

SEQ ID NO: 11 - DNA, 25 bp, Artificial Sequence
  Primer
ttttgcagagttaatcagacacttg

SEQ ID NO: 12 - DNA, 20 bp, Artificial Sequence
  Primer
gtcccacagaaagggttcaa

SEQ ID NO: 13 - DNA, 20 bp, Artificial Sequence
  Primer
caaaggacacagacaagccc

SEQ ID NO: 14 - DNA, 20 bp, Artificial Sequence
  Primer
ccacactcaaccagaacagc

SEQ ID NO: 15 - DNA, 20 bp, Artificial Sequence
  Primer
gtccaactccgtctggaagg

SEQ ID NO: 16 - DNA, 20 bp, Artificial Sequence
  Primer
aagacaggagcaggagagcc

SEQ ID NO: 17 - DNA, 20 bp, Artificial Sequence
  Primer
tgcctggttttaggtttccc

SEQ ID NO: 18 - DNA, 20 bp, Artificial Sequence
  Primer
ctttagtcgcccacactgtg

SEQ ID NO: 19 - DNA, 20 bp, Artificial Sequence
  Primer
caggttcacccaggtctcag

SEQ ID NO: 20 - DNA, 20 bp, Artificial Sequence
  Primer
cagcctggttcaggatgaag

SEQ ID NO: 21 - DNA, 20 bp, Artificial Sequence
  Primer
cacagaggaaggatcgaagg

SEQ ID NO: 22 - DNA, 22 bp, Artificial Sequence
  Primer
ctatcaacatccatgacggggc

SEQ ID NO: 23 - DNA, 20 bp, Artificial Sequence
  Primer
ttggcagaatgtagccagac

SEQ ID NO: 24 - DNA, 20 bp, Artificial Sequence
  Primer
gccttgtcctgctctctttg

SEQ ID NO: 25 - DNA, 20 bp, Artificial Sequence
  Primer
aagtccccacacacctgatg

SEQ ID NO: 26 - DNA, 23 bp, Artificial Sequence
  Primer
gaactacccttacgcatgcaact

SEQ ID NO: 27 - DNA, 23 bp, Artificial Sequence
  Primer
aatattggcacacctccttgatg

SEQ ID NO: 28 - DNA, 23 bp, Artificial Sequence
  Primer
ggacccgagaagacctcctcctt

SEQ ID NO: 29 - DNA, 21 bp, Artificial Sequence
  Primer
tcaatggtgcctctggagatt

SEQ ID NO: 30 - protein, 13 aa, Homo sapiens
STSMEHFRWGKPV

SEQ ID NO: 31 - protein, 12 aa, Homo sapiens
STSMEHFRWGKP